(19) United States
(12) Patent Application Publication
(10) Pub. No.: US 2015/0329772 A1
(43) Pub. Date: Nov. 19, 2015

(54) Materials for Electronic Devices

(71) Applicant: MERCK PATENT GMBH, Darmstadt (DE)

(72) Inventors: Holger Heil, Frankfurt Am Main (DE); Lara-Isabel Rodriguez, Darmstadt (DE); Fabrice Eckes, Darmstadt (DE); Anja Gerhard, Egelsbach (DE)

(73) Assignee: Merck Patent GmbH, Darmstadt (DE)

(21) Appl. No.: 14/758,836

(22) PCT Filed: Dec. 9, 2013
(86) PCT No.: PCT/EP2013/003716

§ 371 Date: Jul. 1, 2015

(30) Foreign Application Priority Data
Jan. 3, 2013 (EP) 13000012.8

(51) Int. Cl. C09K 11/02; H01L 51/00; C07D 307/91; C07C 211/61; C09K 11/06

(52) U.S. Cl. CPC C09K 11/025; C07C 211/61; C09K 11/06; C07D 307/91; H01L 51/006; H01L 51/0061; C09K 2211/1014; C09K 2211/1011; C09K 2211/1007; C09K 2211/1088; C07C 2103/40; C07C 2103/18; C07C 2103/93; H01L 51/0058; H01L 51/0073; H01L 51/5096

(57) ABSTRACT

The present application relates to a compound of a formula (I), which is suitable for use as functional material in an electronic device, in particular as emitter material in an organic electroluminescent device.

The present invention relates to a compound of a formula (I). The compound is suitable for use as functional material in an electronic device, in particular an organic electroluminescent device (OLED). The invention furthermore relates to certain embodiments of electronic devices comprising the compound of the formula (I), and to a process for the preparation of the compound of the formula (I).

In accordance with the present invention, the term electronic device is taken to mean in general electronic devices which comprise organic materials. These are preferably taken to mean OLEDs and some further embodiments of electronic devices comprising organic materials which are disclosed later in the application.

The general structure and principle of functioning of OLEDs are known to the person skilled in the art and is described, inter alia, in U.S. Pat. No. 4,539,507, U.S. Pat. No. 5,151,629, EP 0676461 and WO 1998/27136.

With respect to the performance data of the electronic devices, further improvements are necessary, in particular with a view to broad commercial use, for example in displays or as light sources. Of particular importance in this connection are the lifetime, the efficiency and the operating voltage of the electronic devices and the colour values achieved.

In particular in the case of blue-emitting OLEDs, there is potential for improvement with respect to the lifetime of the devices and the colour values achieved for the emitted light.

An important starting point for achieving the said improvements is the choice of the emitter compound employed in the electronic device.

Blue-fluorescent emitters known from the prior art are a multiplicity of compounds, in particular arylamines containing one or more condensed aryl groups and/or indenofluorene groups. Examples thereof are the pyrene-arylamines disclosed in U.S. Pat. No. 5,153,073 and the pyrene-arylamines disclosed in WO 2012/048780. Further examples of arylamine emitters are benzoindenofluorenamines, for example in accordance with WO 2008/006449, and dibenzoindenofluorenamines, for example in accordance with WO 2007/140847.

Furthermore, the use of fluorenamines which contain aromatic groups condensed onto the fluorene system is known in the prior art. The compounds which contain two or more arylamino groups are employed as fluorescent emitters (US 2012/0161615). However, the compounds exhibit green to green-blue emission and not blue emission.

Furthermore, KR 2009/131536 and WO 2004/061048 disclose benzofluorene derivatives which carry a diphenylamino group. However, compounds of this type have excessively short-wave emission to be used as blue-fluorescent emitters, or their efficiency and lifetime are unsatisfactory on use in OLEDs.

In summary, the technical object is thus to provide deep-blue-fluorescent emitters which preferably have a narrow emission band. The object is furthermore preferably to provide compounds with which high power efficiency and a long lifetime and deep-blue emission of the electronic device can be achieved.

Surprisingly, it has now been found that arylamine compounds in which two or more benzofluorene units are bonded to the nitrogen have deep-blue colour coordinates and a very narrow emission spectrum and consequently achieve the technical object presented above. Deep-blue colour coordinates in the case of emitter compounds are highly desirable for use in displays and lighting applications. In particular in the case of blue-emitting emitter compounds, a narrow emission spectrum, i.e. an emission band having a small width, is highly desirable for tuning the colour impressions of the various colours in a display or in the case of a lighting application.

The invention thus relates to a compound of a formula (I)

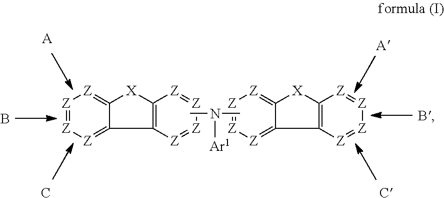

where:
an aromatic or heteroaromatic six-membered ring, which may be substituted by one or more radicals R¹, is condensed onto at least one of the three bonds selected from bonds A, B and C and onto at least one of the three bonds selected from bonds A′, B′ and C′,
Z is on each occurrence, identically or differently, CR¹ or N;
X is on each occurrence, identically or differently, BR², C(R²)₂, C(R²)₂—C(R²)₂, —R²C═CR²—, —R²C═N—, Si(R²)₂, Si(R²)₂—Si(R²)₂, C═O, O, S, S═O, SO₂, NR², PR² or P(═O)R²;
Ar¹ is an aromatic or heteroaromatic ring system having 6 to 40 aromatic ring atoms, which may be substituted by one or more radicals R¹;
R¹ is on each occurrence, identically or differently, H, D, F, Cl, Br, I, C(═O)R³, CN, Si(R³)₃, P(═O)(R³)₂, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R³ and where one or more CH₂ groups in the above-mentioned groups may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂ and where one or more H atoms in the above-mentioned groups may be replaced by D, F, Cl, Br, I or CN, or an aromatic or heteroaromatic ring system having 5 to 30 aromatic ring atoms, which may in each case be substituted by one or more radicals R³, or an aryloxy or heteroaryloxy group having 5 to 30 aromatic ring atoms, which may be substituted by one or more radicals R³, where two or more radicals R¹ may be linked to one another and may form a ring;
R² is on each occurrence, identically or differently, H, D, F, Cl, Br, I, C(═O)R³, CN, Si(R³)₃, N(R³)₂, P(═O)(R³)₂, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R³ and where one or more CH₂ groups in the above-mentioned groups may be replaced by —R³C═CR³—, —C≡C—, Si(R³)₂, C═O, C═NR³, —C(═O)O—, —C(═O)NR³—, NR³, P(═O)(R³), —O—, —S—, SO or SO₂ and where one or more H atoms in the above-mentioned groups may be replaced by D, F, Cl, Br, I or CN, or an aromatic or heteroaromatic ring system having 5 to 30 aromatic ring atoms, which may in each case be substituted by one or more radicals R³, or an aryloxy or heteroaryloxy group having 5 to 30 aromatic ring atoms, which may be substituted by one or more radicals R³, where two or more radicals R² may be linked to one another and may form a ring;
R³ is on each occurrence, identically or differently, H, D, F, Cl, Br, I, C(═O)R⁴, CN, Si(R⁴)₃, N(R⁴)₂, P(═O)(R⁴)₂, S(═O)R⁴, S(═O)₂R⁴, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where the above-mentioned groups may each be substituted by one or more radicals R⁴ and where one or more CH₂ groups in the above-mentioned groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂ and where one or more H atoms in the above-mentioned groups may be replaced by D, F, Cl, Br, I or CN, or an aromatic or heteroaromatic ring system having 5 to 30 aromatic ring atoms, which may in each case be substituted by one or more radicals R⁴, or an aryloxy or heteroaryloxy group having 5 to 30 aromatic ring atoms, which may be substituted by one or more radicals R⁴, where two or more radicals R¹ may be linked to one another and may form a ring;
R⁴ is on each occurrence, identically or differently, H, D, F or an aliphatic, aromatic or heteroaromatic organic radical having 1 to 20 C atoms, in which, in addition, one or more H atoms may be replaced by D or F; two or more substituents R⁴ here may be linked to one another and may form a ring;
   where the case in which an aromatic or heteroaromatic six-membered ring is condensed onto each of the two bonds A and A′ and no further aromatic or heteroaromatic six-membered ring is condensed onto one of the other four bonds selected from bonds B, C, B′ and C′ is excluded.

In the formula (I), bonds A, B, C, A′, B′ and C′ are denoted by arrows for better clarity.

The formulation that an aromatic or heteroaromatic six-membered ring is condensed onto a bond, such as, for example, bond A, is, for the purposes of the present application, taken to mean that the following structure forms:

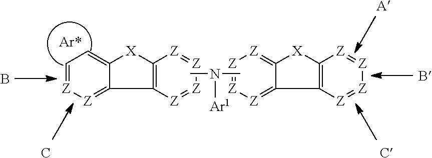

where Ar* is an aromatic or heteroaromatic six-membered ring which, as shown above, contains the two ring atoms of bond A.

In the case where Ar* represents an unsubstituted phenyl group, the following structure, for example, is then present:

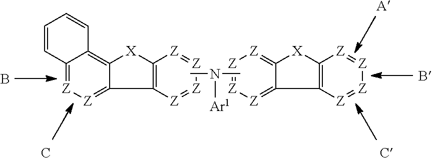

The following are general definitions of chemical groups for the purposes of the present application:

An aryl group in the sense of this invention contains 6 to 60 aromatic ring atoms; a heteroaryl group in the sense of this invention contains 5 to 60 aromatic ring atoms, at least one of which is a heteroatom. The heteroatoms are preferably selected from N, O and S. This represents the basic definition. If other preferences are indicated in the description of the present invention, for example with respect to the number of aromatic ring atoms or the heteroatoms present, these apply.

An aryl group or heteroaryl group here is taken to mean either a simple aromatic ring, i.e. benzene, or a simple heteroaromatic ring, for example pyridine, pyrimidine or thiophene, or a condensed (annellated) aromatic or heteroaromatic polycycle, for example naphthalene, phenanthrene, quinoline or carbazole. A condensed (annellated) aromatic or heteroaromatic polycycle in the sense of the present application consists of two or more simple aromatic or heteroaromatic rings condensed with one another.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aryloxy group in accordance with the definition of the present invention is taken to mean an aryl group, as defined above, which is bonded via an oxygen atom. An analogous definition applies to heteroaryloxy groups.

An aromatic ring system in the sense of this invention contains 6 to 60 C atoms in the ring system. A heteroaromatic ring system in the sense of this invention contains 5 to 60 aromatic ring atoms, at least one of which is a heteroatom. The heteroatoms are preferably selected from N, O and/or S. An aromatic or heteroaromatic ring system in the sense of this invention is intended to be taken to mean a system which does not necessarily contain only aryl or heteroaryl groups, but instead in which, in addition, a plurality of aryl or heteroaryl groups may be connected by a non-aromatic unit (preferably less than 10% of the atoms other than H), such as, for example, an sp³-hybridised C, Si, N or O atom, an sp²-hybridised C or N atom or an sphybridised C atom. Thus, for example, systems such as 9,9′-spirobifluorene, 9,9′-diarylfluorene, triarylamine, diaryl ether, stilbene, etc., are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl or heteroaryl groups are linked to one another via single bonds are also taken to be aromatic or heteroaromatic ring systems in the sense of this invention, such as, for example, systems such as biphenyl, terphenyl or diphenyltriazine.

An aromatic or heteroaromatic ring system having 5-60 aromatic ring atoms, which may in each case also be substituted by radicals as defined above and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenylene, quaterphenyl, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, indolocarbazole, indenocarbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyndazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or combinations of these groups.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluoromethoxy, ethoxy, npropoxy, i-propoxy, n-butoxy, i-butoxy, s-butoxy, t-butoxy, n-pentoxy, spentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyciohexylthio, n-heptylthio, cycloheptylthio, n-octylthio, cyclooctylthio, 2-ethylhexytthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynyfthio or octynylthio.

The formulation that two or more radicals may form a ring with one another is intended, for the purposes of the present application, to be taken to mean, inter alia, that the two radicals are linked to one another by a chemical bond. This is illustrated by the following scheme:

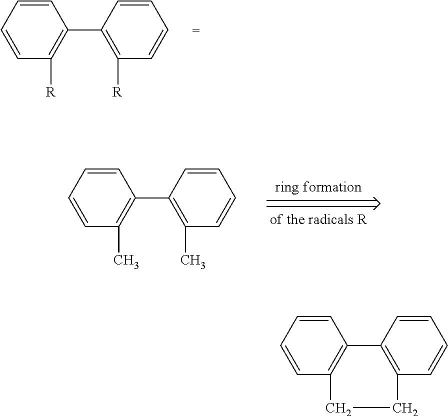

Furthermore, however, the above-mentioned formulation is also intended to be taken to mean that, in the case where one of the two radicals represents hydrogen, the second radical is bonded to the position to which the hydrogen atom was bonded, with formation of a ring. This is intended to be illustrated by the following scheme:

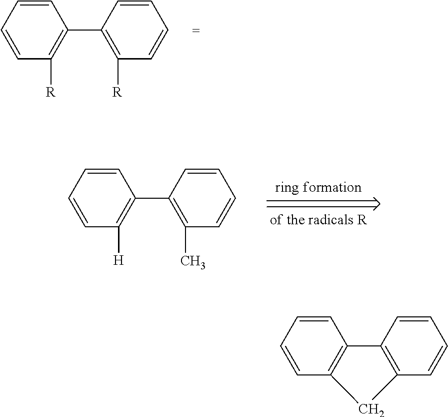

It is preferred for an aromatic or heteroaromatic six-membered ring to be condensed onto neither of the two bonds A and A′ in formula (I).

It is furthermore preferred for an aromatic or heteroaromatic six-membered ring, particularly preferably an aromatic six-membered ring, in each case to be condensed onto at least one of the two bonds selected from bonds B and C and onto at least one of the two bonds selected from bonds B′ and C′, where the aromatic or heteroaromatic six-membered ring may be substituted by one or more radicals R¹.

For compounds of the formula (I), it is preferred for the group X to be selected on each occurrence, identically or differently, from BR², C(R²)₂, Si(R²)₂, O, S and NR². X is particularly preferably equal to C(R²)₂.

It is furthermore preferred in the case of compounds of the formula (I) for not more than three groups Z per six-membered ring to be equal to N. Furthermore preferably, not more than two adjacent groups Z are equal to N.

It is generally preferred for compounds of the formula (I) for Z to be equal to CR¹.

It is furthermore preferred for Ar¹ to be selected from aromatic or heteroaromatic ring systems having 6 to 24 aromatic ring atoms, which may be substituted by one or more radicals R¹. Ar¹ is particularly preferably selected from aromatic ring systems having 6 to 18 aromatic ring atoms, which may be substituted by one or more radicals R¹. Ar¹ is very particularly preferably selected from phenyl, naphthyl, biphenyl, terphenyl, fluorenyl, monobenzofluorenyl, dibenzofluorenyl, spirobifluorenyl, indenofluorenyl, dibenzofuranyl, carbazolyl and dibenzothiophenyl, each of which may be substituted by one or more radicals R¹.

It is furthermore preferred for Ar¹ not to represent a group of the formula

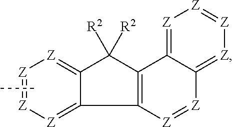

where Z, R¹ and R² are defined as indicated above in general terms for formula (I); and
where the dashed line denotes the bond to the nitrogen atom.

Preferred groups Ar¹ are the groups of the formulae (Ar¹-1) to (Ar¹-34) depicted below:

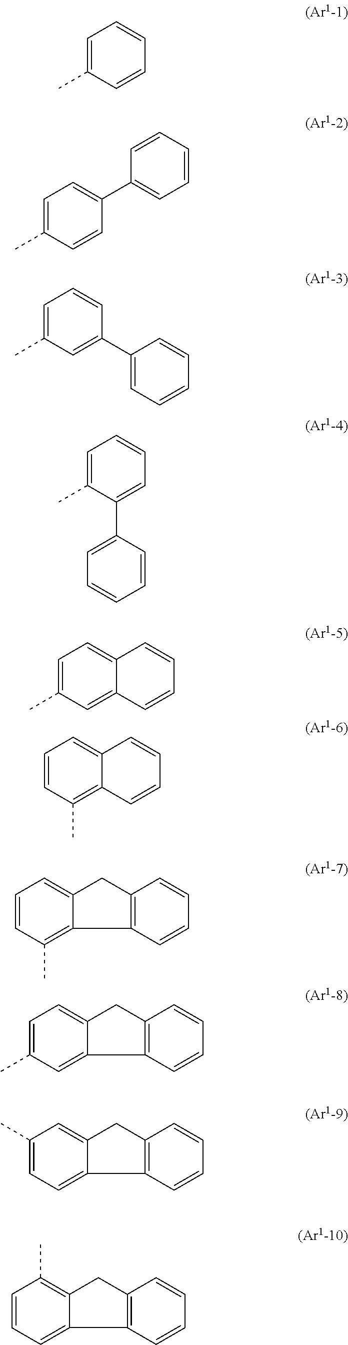

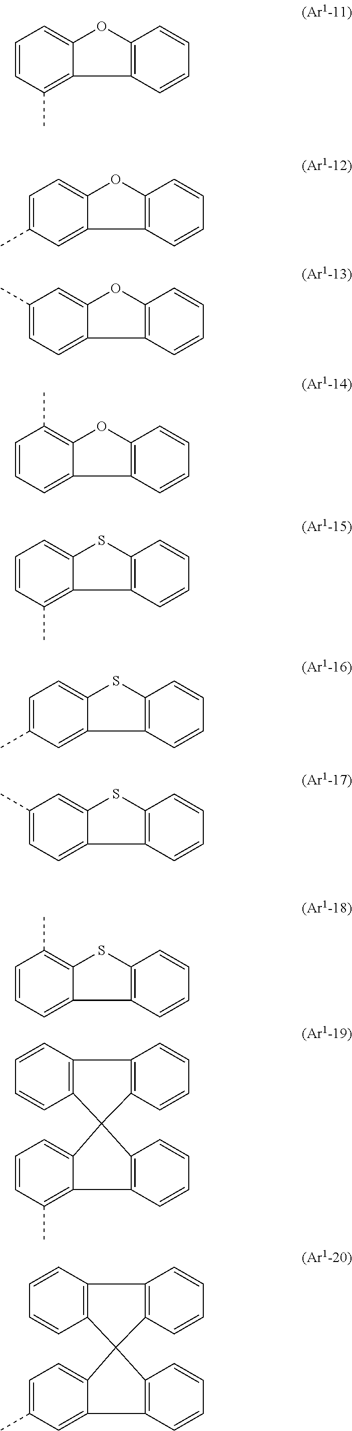

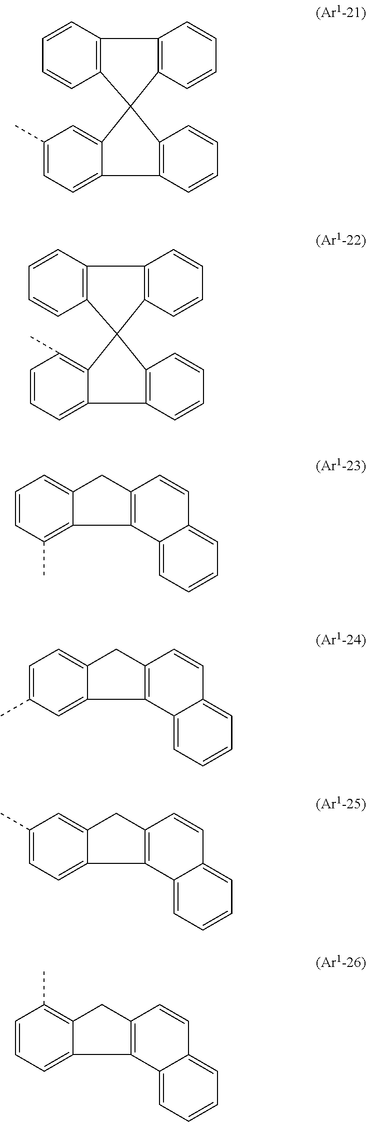

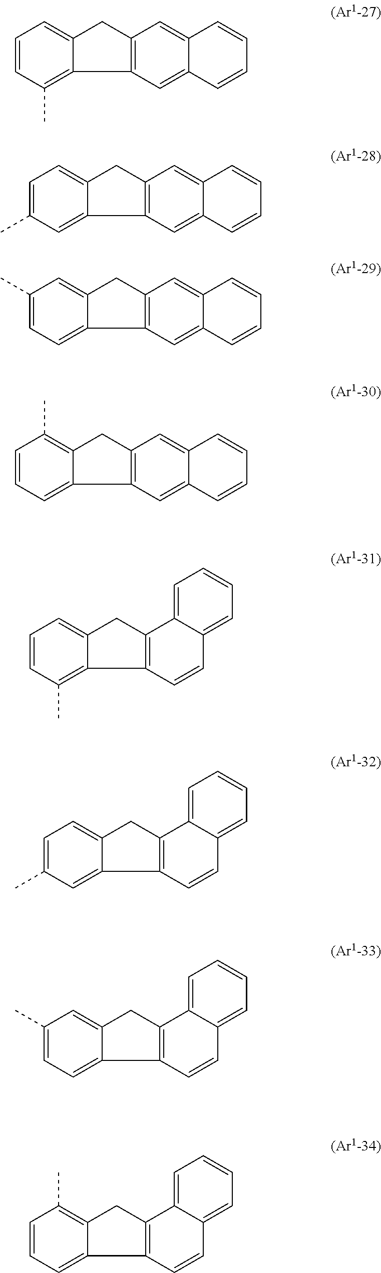

where the dashed line denotes the bond to the remainder of the compound, and the groups may be substituted at free positions by a radical R¹.

R¹ and R² are furthermore preferably on each occurrence, identically or differently, H, D, F, CN, Si(R³)₃, a straight-chain alkyl or alkoxy group having 1 to 20 C atoms or a branched or cyclic alkyl or alkoxy group having 3 to 20 C atoms, where the above-mentioned groups may each be substituted by one or more radicals R³ and where one or more CH₂ groups in the above-mentioned groups may be replaced by —C≡C—, —R³C═CR³—, Si(R³)₂, C═O, C═NR³, —NR³—, —O—, —S—, —C(═O)O— or —C(═O)NR³—, or an aromatic or heteroaromatic ring system having 5 to 20 aromatic ring atoms, which may in each case be substituted by one or more radicals R³, where two or more radicals R¹ or R² may be linked to one another and may form a ring.

R² is particularly preferably selected from straight-chain alkyl groups having 1 to 10 C atoms or branched or cyclic alkyl groups having 3 to 10 C atoms, where the above-mentioned groups may each be substituted by one or more radicals R³, or from aromatic or heteroaromatic ring systems having 5 to 20 aromatic ring atoms, which may in each case be substituted by one or more radicals R³.

It is furthermore preferred for radicals R² which are bonded to the same atom of group X, for example the two radicals R² of a group C(R²)₂, to be connected to one another to form a ring. This is preferably a five-membered ring or a six-membered ring, where the groups are preferably aryl groups or alkyl groups. In these cases, the group X is very particularly preferably selected from groups of the following formulae (X-1) to (X-8)

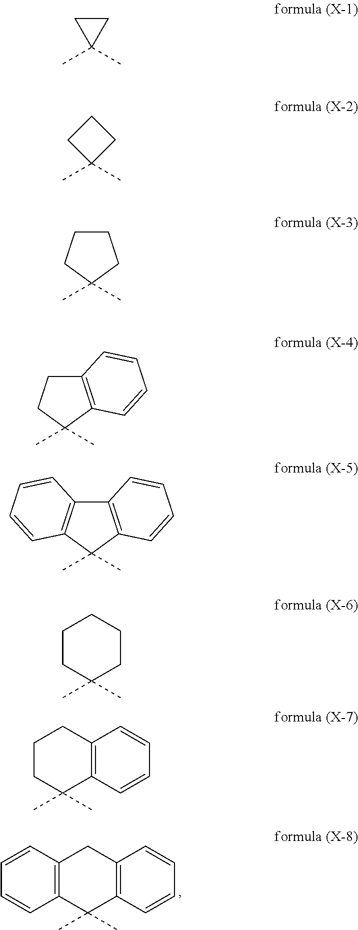

where the groups depicted may optionally be substituted by one or more radicals R³ in the positions shown as unsubstituted, and where the dashed lines represent bonds to the remainder of the compound.

R³ is furthermore preferably on each occurrence, identically or differently, H, D, F, CN, Si(R⁴)₃, a straight-chain alkyl or alkoxy group having 1 to 20 C atoms or a branched or cyclic alkyl or alkoxy group having 3 to 20 C atoms, where the above-mentioned groups may each be substituted by one or more radicals R⁴ and where one or more CH² groups in the above-mentioned groups may be replaced by —C≡C—, —R⁴C═CR⁴—, Si(R⁴)₂, C═O, C═NR⁴, —NR⁴—, —O—, —S—, —C(═O)O— or —C(═O)NR⁴—, or an aromatic or heteroaromatic ring system having 5 to 20 aromatic ring atoms, which may in each case be substituted by one or more radicals R⁴, where two or more radicals R³ may be linked to one another and may form a ring.

It is furthermore preferred for the bonding position of the group NAr¹ to the fluorenyl groups to be in the para-position to the direct bond between the two aromatic six-membered rings of the fluorenyl skeleton. Correspondingly, compounds of the following formula (I-A) are preferred:

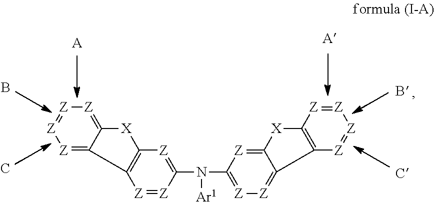

where the groups occurring are as defined above,
where an aromatic or heteroaromatic six-membered ring, which may be substituted by one or more radicals R¹, is condensed onto at least one of the three bonds selected from bonds A, B and C and onto at least one of the three bonds selected from bonds A′, B′ and C′,
where the case in which an aromatic or heteroaromatic six-membered ring is condensed onto each of the two bonds A and A′ and no further aromatic or heteroaromatic six-membered ring is condensed onto one of the other four bonds selected from bonds B, C, B′ and C′ is excluded.

The preferred embodiments of groups indicated above are also regarded as preferred for this embodiment.

In particular, X in compounds of the formula (I-A) is preferably selected on each occurrence, identically or differently, from BR², C(R²)₂, Si(R²)₂, O, S and NR², X is particularly preferably equal to C(R²)₂.

It is furthermore preferred for at least one group R¹ in the compound to be selected from straight-chain alkyl groups having 1 to 10 C atoms or branched or cyclic alkyl groups having 3 to 10 C atoms, which may be substituted by one or more radicals R³, or aromatic or heteroaromatic ring systems having 5 to 20 aromatic ring atoms, preferably from aromatic ring systems having 6 to 20 aromatic ring atoms, where the ring systems may in each case be substituted by one or more radicals R³.

It is furthermore preferred for at least one group R¹ which is bonded to one of the outer rings of the fluorenyl group to be selected from straight-chain alkyl groups having 1 to 10 C atoms or branched or cyclic alkyl groups having 3 to 10 C atoms, which may be substituted by one or more radicals R³, or aromatic or heteroaromatic ring systems having 6 to 18 aromatic ring atoms, which may in each case be substituted by one or more radicals R³. “Outer ring of the fluorenyl group” is taken to mean that ring of the two rings of a fluorenyl group which is not bonded to the group NAr¹. This is particularly preferred if Ar¹ does not represent a benzofluorene group, in particular if Ar¹ is not selected from groups of the formulae (Ar¹-23) to (Ar¹-34).

If Ar¹ is a benzofluorene group, in particular a group selected from groups of the formulae (Ar¹-23) to (Ar¹-34), it may be preferred for the compound to contain no group R¹ which represents an alkyl group or an aromatic or heteroaromatic ring system. It may furthermore be preferred in this case for all groups R¹ to be equal to H.

By means of this substitution pattern, particularly advantageous emission properties of the compound according to the invention are achieved.

The said group R¹ is particularly preferably bonded to the fluorenyl skeleton in the para-position to the direct bond between the two aromatic six-membered rings of the fluorenyl skeleton, so that the compounds of the formula (I) correspond to the following structure of the formula (I-B):

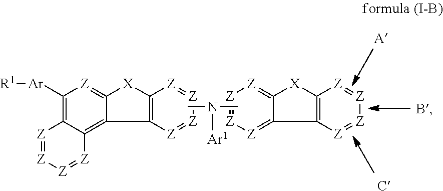

where the groups occurring are as defined above, and
R¹ is selected from straight-chain alkyl groups having 1 to 10 C atoms or branched or cyclic alkyl groups having 3 to 10 C atoms, which may be substituted by one or more radicals R³, or aromatic or heteroaromatic ring systems having 6 to 18 aromatic ring atoms, which may be substituted by one or more radicals R³; and
   where an aromatic or heteroaromatic six-membered ring, which may be substituted by one or more radicals R¹, is condensed onto at least one of the three bonds selected from bonds A′, B′ and C′.

Very particularly preferred groups R^{1-Ar}, as defined above, are selected from methyl, ethyl, propyl, butyl, isopropyl, isobutyl, tert-butyl, cyclohexyl, phenyl, naphthyl, biphenyl, terphenyl, fluorenyl, monobenzofluorenyl, dibenzofluorenyl, spirobifluorenyl, indenofluorenyl, dibenzofuranyl, carbazolyl and dibenzothiophenyl, each of which may be substituted by one or more radicals R³.

Preferred groups R^{1-Ar} are the groups of the formulae (R^{1-Ar}-1) to (R^{1-Ar}-27) depicted below:

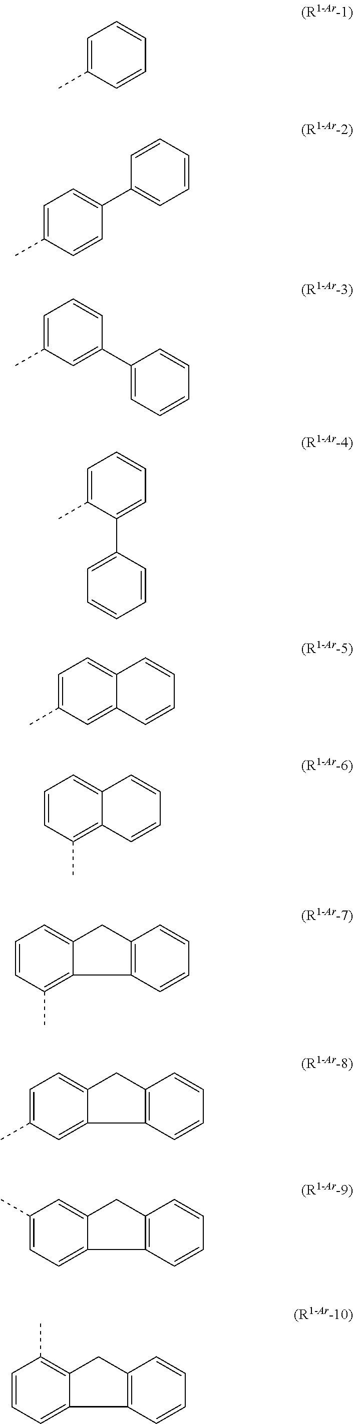

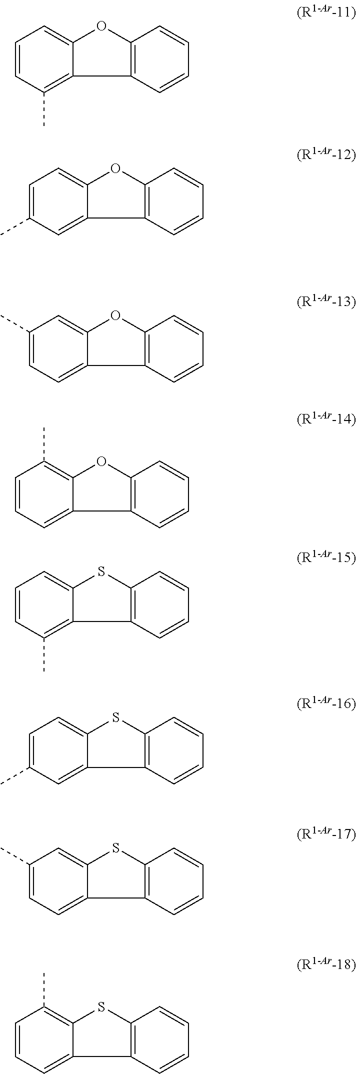

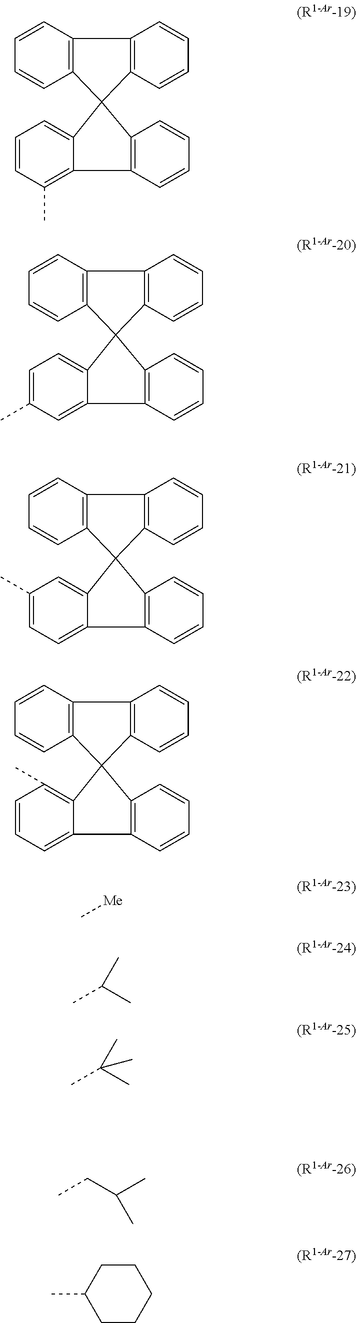

where the dashed line denotes the bond to the remainder of the compound, and the groups may be substituted at free positions by a radical R³.

The compound of the formula (I) furthermore preferably contains no condensed aryl or heteroaryl group having more than 18 aromatic ring atoms in the condensed group. It particularly preferably contains no condensed aryl or heteroaryl group having more than 14 aromatic ring atoms in the condensed group, very particularly preferably no condensed aryl or heteroaryl group having more than 10 aromatic ring atoms in the condensed group.

The compound of the formula (I) furthermore preferably contains no further arylamino group in addition to the arylamino group —N(Ar¹)—. An arylamino group here is taken to mean a group which contains at least one aryl or heteroaryl group bonded to an amino nitrogen atom. The compound of the formula (I) particularly preferably contains no further amino group in addition to the arylamino group —N(Ar¹)—.

Particularly preferred embodiments of compounds of the formula (I) conforms to the following formulae (I-1) to (I-5)

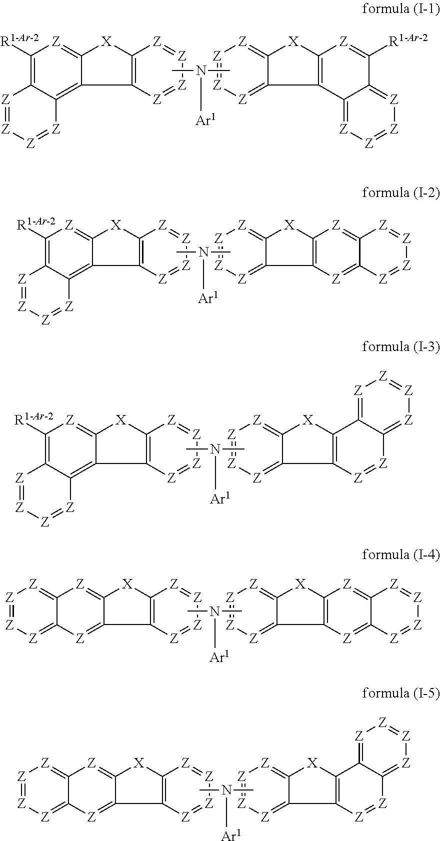

where the groups occurring are defined as above, and where
R^{1-Ar-2} is selected on each occurrence, identically or differently, from H or straight-chain alkyl groups having 1 to 10 C atoms or branched or cyclic alkyl groups having 3 to 10 C atoms, which may be substituted by one or more radicals R³, or aromatic or heteroaromatic ring systems having 6 to 18 aromatic ring atoms, which may be substituted by one or more radicals R³.

Of the above formulae, very particular preference is given to formula (I-1).

The same embodiments of groups as indicated above in general terms for compounds of the formula (I) are regarded as preferred for the compounds of the formulae (I-1) to (I-5). In particular, X is preferably C(R²)₂. The group NAr¹ is furthermore particularly preferably bonded in the para-position to the direct bond between the two aromatic six-membered rings of the fluorene skeleton, corresponding to the embodiment shown in formula (I-A).

Furthermore, R^{1-Ar-2} is preferably selected from H and the formulae (R^{1-Ar}-1) to (R^{1-Ar}-27).

Particularly preferred embodiments of compounds of the formula (I) correspond to the formulae indicated in the following table, where the basic structures are those of the formulae (I-1) to (I-5), and the groups occurring are defined as above.

| Formula (I- | Basic structure | Ar¹ |
|---|---|---|
| 1-1) | Formula (I-1) | (Ar¹-1) |
| 1-2) | ″ | (Ar¹-2) |
| 1-3) | ″ | (Ar¹-3) |
| 1-4) | ″ | (Ar¹-4) |
| 1-5) | ″ | (Ar¹-5) |
| 1-6) | ″ | (Ar¹-6) |
| 1-7) | ″ | (Ar¹-7) |
| 1-8) | ″ | (Ar¹-8) |
| 1-9) | ″ | (Ar¹-9) |
| 1-10) | ″ | (Ar¹-10) |
| 1-11) | ″ | (Ar¹-11) |
| 1-12) | ″ | (Ar¹-12) |
| 1-13) | ″ | (Ar¹-13) |
| 1-14) | ″ | (Ar¹-14) |
| 1-15) | ″ | (Ar¹-15) |
| 1-16) | ″ | (Ar¹-16) |
| 1-17) | ″ | (Ar¹-17) |
| 1-18) | ″ | (Ar¹-18) |
| 1-19) | ″ | (Ar¹-19) |
| 1-20) | ″ | (Ar¹-20) |
| 1-21) | ″ | (Ar¹-21) |
| 1-22) | ″ | (Ar¹-22) |
| 1-23) | ″ | (Ar¹-23) |
| 1-24) | ″ | (Ar¹-24) |
| 1-25) | ″ | (Ar¹-25) |
| 1-26) | ″ | (Ar¹-26) |
| 1-27) | ″ | (Ar¹-27) |
| 1-28) | ″ | (Ar¹-28) |
| 1-29) | ″ | (Ar¹-29) |
| 1-30) | ″ | (Ar¹-30) |
| 1-31) | ″ | (Ar¹-31) |
| 1-32) | ″ | (Ar¹-32) |
| 1-33) | ″ | (Ar¹-33) |
| 1-34) | ″ | (Ar¹-34) |
| 2-1) | Formula (I-2) | (Ar¹-1) |
| 2-2) | ″ | (Ar¹-2) |
| 2-3) | ″ | (Ar¹-3) |
| 2-4) | ″ | (Ar¹-4) |
| 2-5) | ″ | (Ar¹-5) |
| 2-6) | ″ | (Ar¹-6) |
| 2-7) | ″ | (Ar¹-7) |
| 2-8) | ″ | (Ar¹-8) |
| 2-9) | ″ | (Ar¹-9) |
| 2-10) | ″ | (Ar¹-10) |
| 2-11) | ″ | (Ar¹-11) |
| 2-12) | ″ | (Ar¹-12) |
| 2-13) | ″ | (Ar¹-13) |
| 2-14) | ″ | (Ar¹-14) |
| 2-15) | ″ | (Ar¹-15) |
| 2-16) | ″ | (Ar¹-16) |
| 2-17) | ″ | (Ar¹-17) |
| 2-18) | ″ | (Ar¹-18) |
| 2-19) | ″ | (Ar¹-19) |
| 2-20) | ″ | (Ar¹-20) |
| 2-21) | ″ | (Ar¹-21) |
| 2-22) | ″ | (Ar¹-22) |
| 2-23) | ″ | (Ar¹-23) |
| 2-24) | ″ | (Ar¹-24) |
| 2-25) | ″ | (Ar¹-25) |
| 2-26) | ″ | (Ar¹-26) |
| 2-27) | ″ | (Ar¹-27) |
| 2-28) | ″ | (Ar¹-28) |
| 2-29) | ″ | (Ar¹-29) |
| 2-30) | ″ | (Ar¹-30) |
| 2-31) | ″ | (Ar¹-31) |
| 2-32) | ″ | (Ar¹-32) |
| 2-33) | ″ | (Ar¹-33) |
| 2-34) | ″ | (Ar¹-34) |
| 3-1) | Formula (I-3) | (Ar¹-1) |
| 3-2) | ″ | (Ar¹-2) |
| 3-3) | ″ | (Ar¹-3) |
| 3-4) | ″ | (Ar¹-4) |
| 3-5) | ″ | (Ar¹-5) |
| 3-6) | ″ | (Ar¹-6) |
| 3-7) | ″ | (Ar¹-7) |
| 3-8) | ″ | (Ar¹-8) |
| 3-9) | ″ | (Ar¹-9) |
| 3-10) | ″ | (Ar¹-10) |
| 3-11) | ″ | (Ar¹-11) |
| 3-12) | ″ | (Ar¹-12) |
| 3-13) | ″ | (Ar¹-13) |
| 3-14) | ″ | (Ar¹-14) |
| 3-15) | ″ | (Ar¹-15) |
| 3-16) | ″ | (Ar¹-16) |
| 3-17) | ″ | (Ar¹-17) |
| 3-18) | ″ | (Ar¹-18) |
| 3-19) | ″ | (Ar¹-19) |
| 3-20) | ″ | (Ar¹-20) |
| 3-21) | ″ | (Ar¹-21) |
| 3-22) | ″ | (Ar¹-22) |
| 3-23) | ″ | (Ar¹-23) |
| 3-23) | ″ | (Ar¹-23) |
| 3-24) | ″ | (Ar¹-24) |
| 3-25) | ″ | (Ar¹-25) |
| 3-26) | ″ | (Ar¹-26) |
| 3-27) | ″ | (Ar¹-27) |
| 3-28) | ″ | (Ar¹-28) |
| 3-29) | ″ | (Ar¹-29) |
| 3-30) | ″ | (Ar¹-30) |
| 3-31) | ″ | (Ar¹-31) |
| 3-32) | ″ | (Ar¹-32) |
| 3-33) | ″ | (Ar¹-33) |
| 3-34) | ″ | (Ar¹-34) |
| 4-1) | Formula (I-4) | (Ar¹-1) |
| 4-2) | ″ | (Ar¹-2) |
| 4-3) | ″ | (Ar¹-3) |
| 4-4) | ″ | (Ar¹-4) |
| 4-5) | ″ | (Ar¹-5) |
| 4-6) | ″ | (Ar¹-6) |
| 4-7) | ″ | (Ar¹-7) |
| 4-8) | ″ | (Ar¹-8) |
| 4-9) | ″ | (Ar¹-9) |
| 4-10) | ″ | (Ar¹-10) |
| 4-11) | ″ | (Ar¹-11) |
| 4-12) | ″ | (Ar¹-12) |
| 4-13) | ″ | (Ar¹-13) |
| 4-14) | ″ | (Ar¹-14) |
| 4-15) | ″ | (Ar¹-15) |
| 4-16) | ″ | (Ar¹-16) |
| 4-17) | ″ | (Ar¹-17) |
| 4-18) | ″ | (Ar¹-18) |
| 4-19) | ″ | (Ar¹-19) |
| 4-20) | ″ | (Ar¹-20) |
| 4-21) | ″ | (Ar¹-21) |
| 4-22) | ″ | (Ar¹-22) |
| 4-23) | ″ | (Ar¹-23) |
| 4-24) | ″ | (Ar¹-24) |
| 4-25) | ″ | (Ar¹-25) |
| 4-26) | ″ | (Ar¹-26) |
| 4-27) | ″ | (Ar¹-27) |
| 4-28) | ″ | (Ar¹-28) |
| 4-29) | ″ | (Ar¹-29) |
| 4-30) | ″ | (Ar¹-30) |
| 4-31) | ″ | (Ar¹-31) |
| 4-32) | ″ | (Ar¹-32) |
| 4-33) | ″ | (Ar¹-33) |
| 4-34) | ″ | (Ar¹-34) |
| 5-1) | Formula (I-5) | (Ar¹-1) |
| 5-2) | ″ | (Ar¹-2) |
| 5-3) | ″ | (Ar¹-3) |
| 5-4) | ″ | (Ar¹-4) |
| 5-5) | ″ | (Ar¹-5) |
| 5-6) | ″ | (Ar¹-6) |
| 5-7) | ″ | (Ar¹-7) |
| 5-8) | ″ | (Ar¹-8) |
| 5-9) | ″ | (Ar¹-9) |
| 5-10) | ″ | (Ar¹-10) |
| 5-11) | ″ | (Ar¹-11) |
| 5-12) | ″ | (Ar¹-12) |
| 5-13) | ″ | (Ar¹-13) |
| 5-14) | ″ | (Ar¹-14) |
| 5-15) | ″ | (Ar¹-15) |
| 5-16) | ″ | (Ar¹-16) |
| 5-17) | ″ | (Ar¹-17) |
| 5-18) | ″ | (Ar¹-18) |
| 5-19) | ″ | (Ar¹-19) |
| 5-20) | ″ | (Ar¹-20) |
| 5-21) | ″ | (Ar¹-21) |
| 5-22) | ″ | (Ar¹-22) |
| 5-23) | ″ | (Ar¹-23) |
| 5-25) | ″ | (Ar¹-24) |
| 5-25) | ″ | (Ar¹-25) |
| 5-26) | ″ | (Ar¹-26) |
| 5-27) | ″ | (Ar¹-27) |
| 5-28) | ″ | (Ar¹-28) |
| 5-29) | ″ | (Ar¹-29) |
| 5-30) | ″ | (Ar¹-30) |
| 5-31) | ″ | (Ar¹-31) |
| 5-32) | ″ | (Ar¹-32) |
| 5-33) | ″ | (Ar¹-33) |
| 5-34) | ″ | (Ar¹-34) |

For compounds from the above table, R^{1-Ar-2} is preferably selected from H and the formulae (R^{1-Ar}-1) to (R^{1-Ar}-27).

The following compounds are examples of compounds of the formula (I):

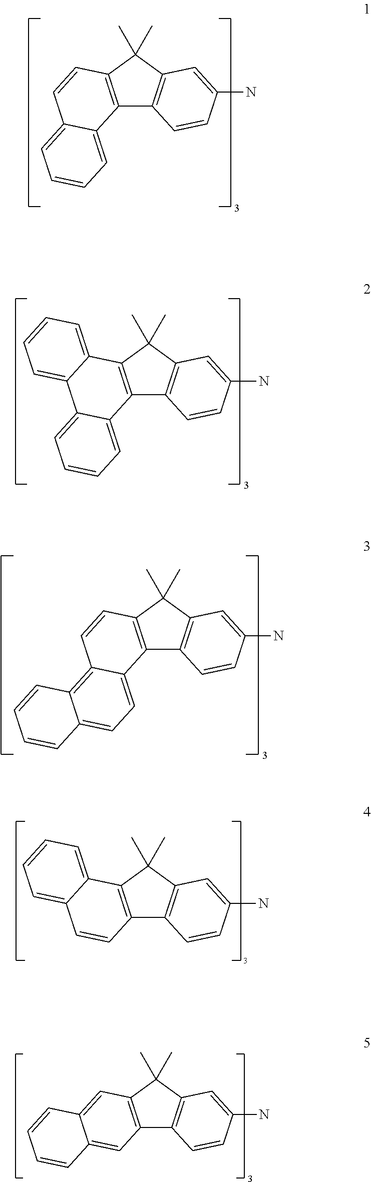

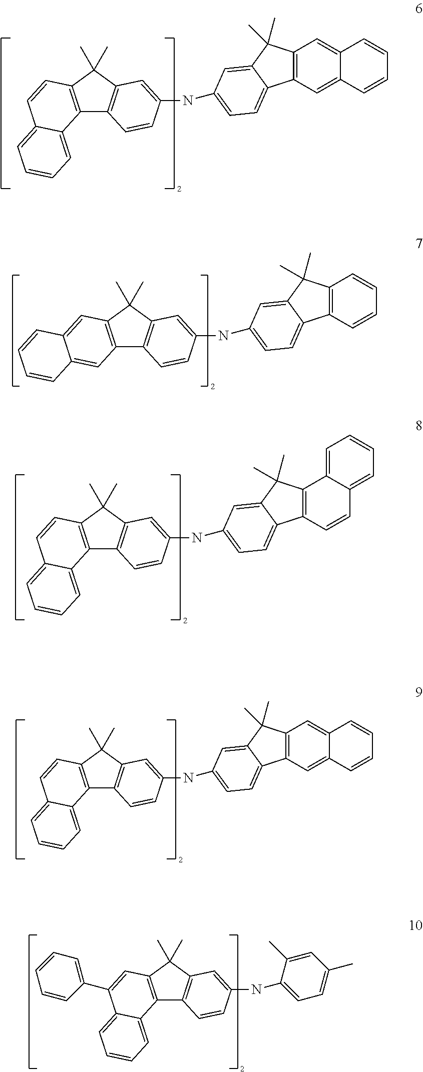

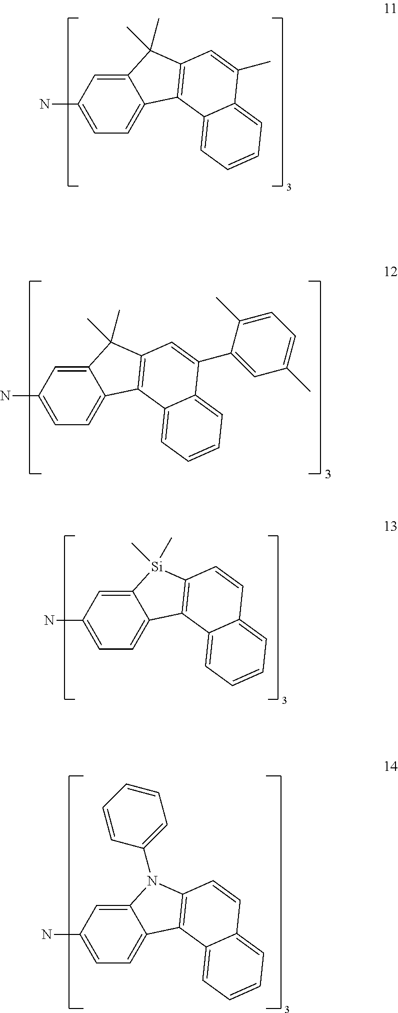

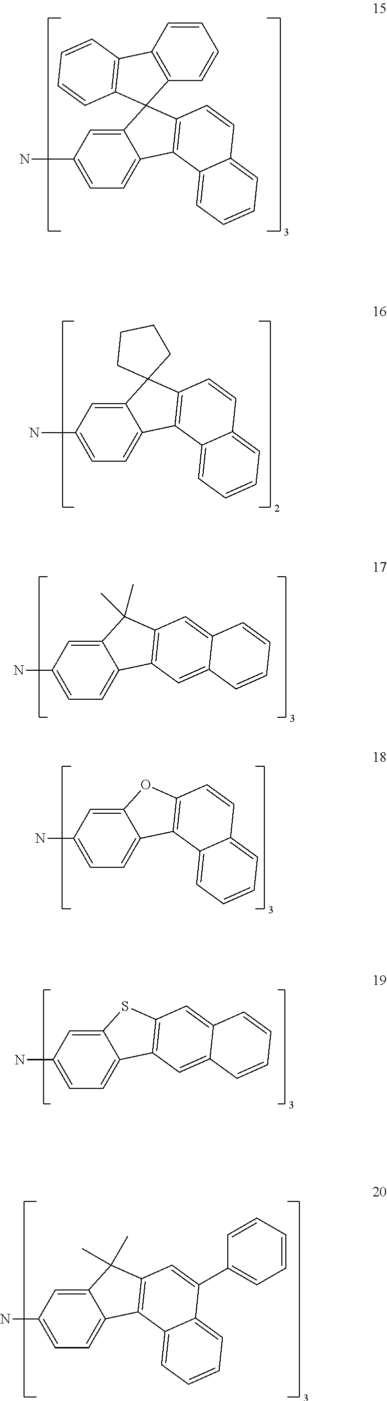

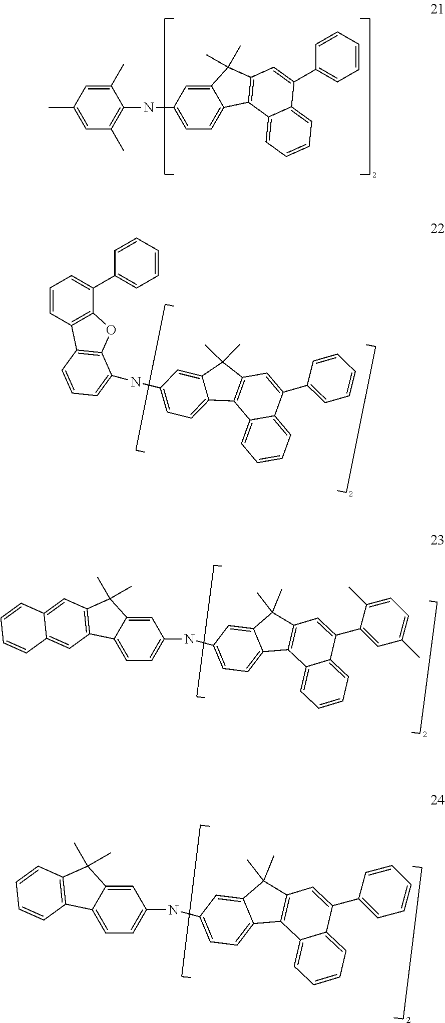

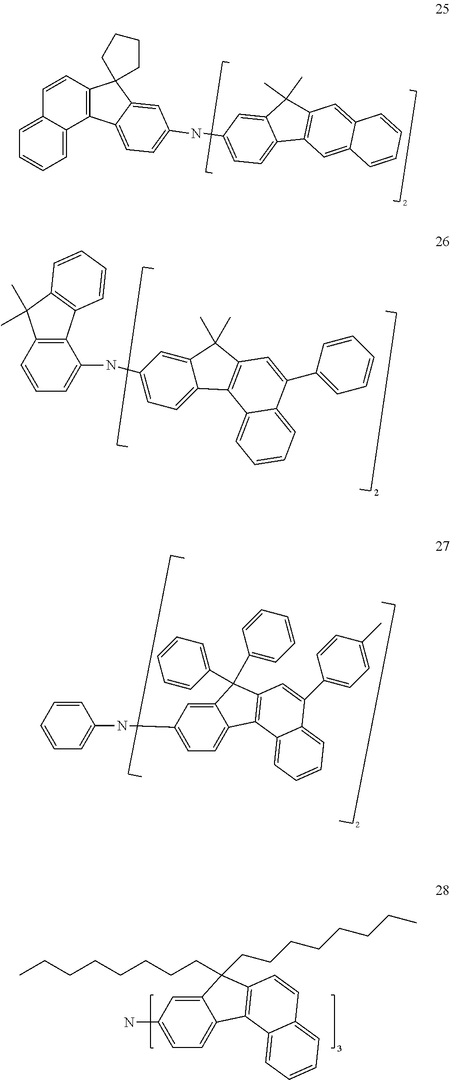

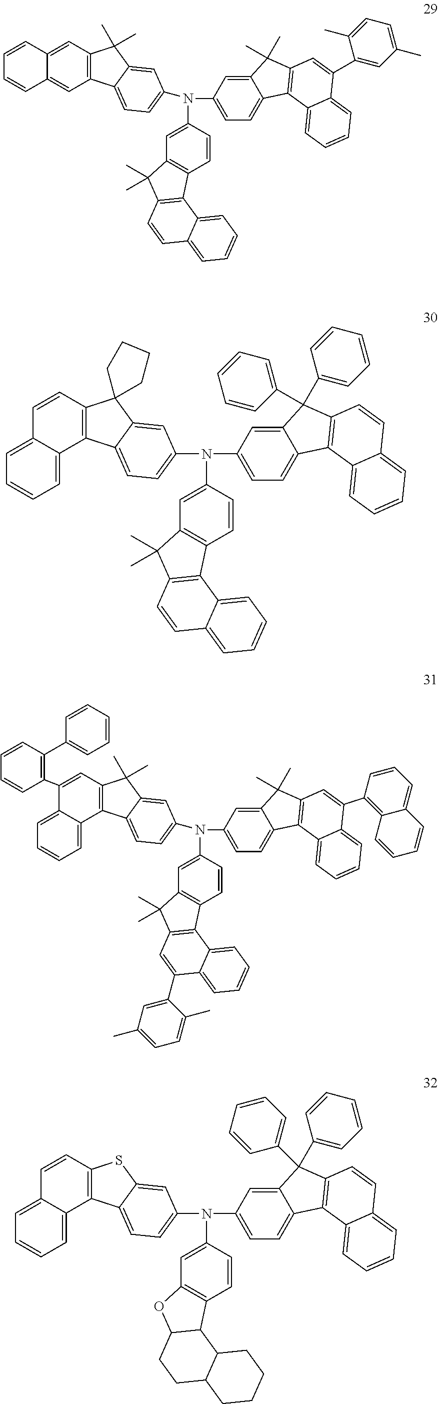

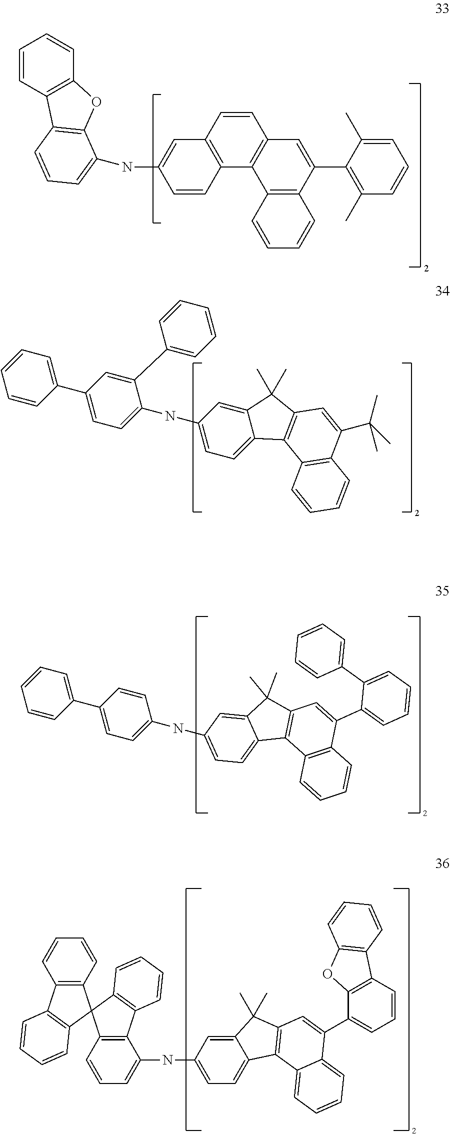

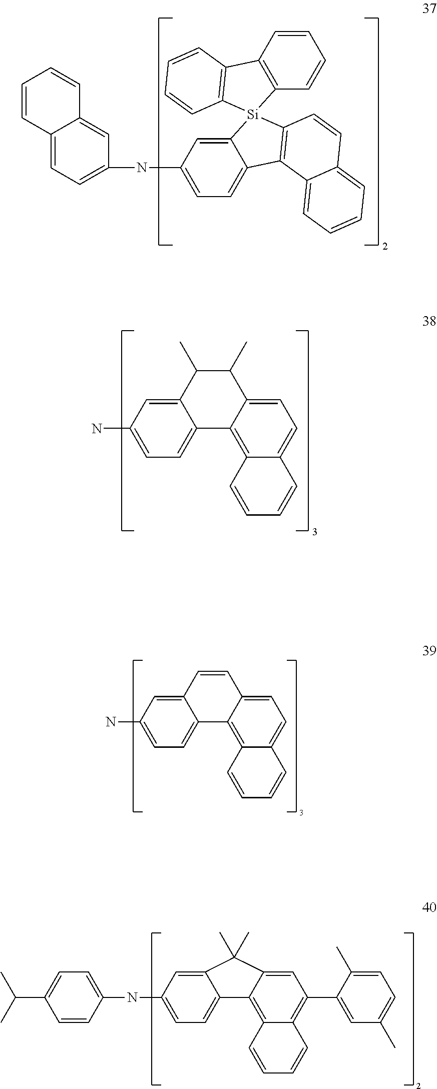

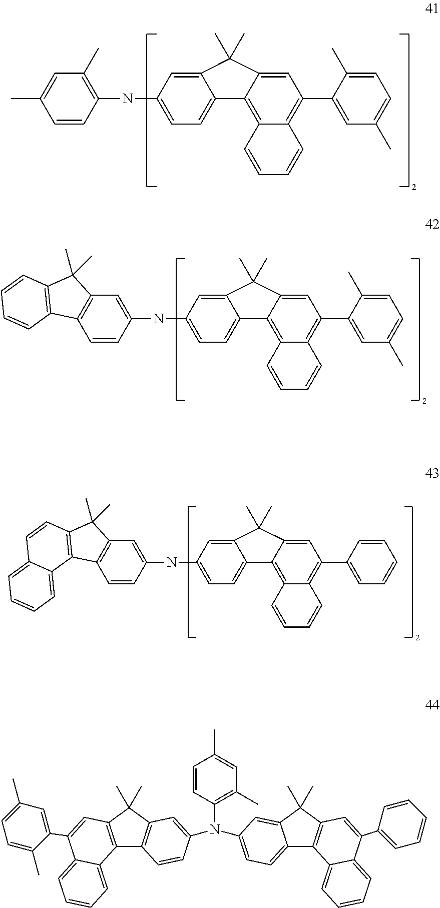

The present application furthermore relates to a process for the preparation of a compound according to the invention.

Preference is given to a process for the preparation of a compound according to the invention, characterised in that it includes at least one transition metal-catalysed coupling reaction. The coupling reaction is preferably selected from Buchwald coupling reactions and Suzuki coupling reactions. Furthermore and preferably in combination with the above-mentioned preferred embodiment of the process, the process includes one or more ring-closure reactions, particularly preferably acid-induced ring-closure reactions of tertiary alcohols to give methylene bridges.

Particularly preferred embodiments of processes for the preparation of compounds according to the invention are described below. Possible synthetic processes are thus proposed to the person skilled in the art with which he will be able to prepare the compounds according to the invention. However, the person skilled in the art is not tied to the processes presented. He will be able to modify and adapt them as necessary within the bounds of his general expert knowledge, if this is necessary.

The compounds according to the invention can be prepared using known synthetic steps of organic chemistry. Preferred reactions here are cyclisation reactions, Buchwald couplings and Suzuki couplings.

Schemes 1 to 3 below show three different routes for the preparation of compounds according to the invention. The process according to Scheme 1 is particularly suitable for the synthesis of compounds according to the invention which contain an aryl group bonded to the benzofluorenyl group. The process according to Scheme 3 is particularly suitable for the preparation of compounds according to the invention which contain three benzofluorene groups.

In Schemes 1 to 3, Ar denotes any desired aryl or heteroaryl group, and Hal denotes any desired reactive group, preferably a halogen group, particularly preferably bromine. All compounds shown may optionally be substituted by one or more organic radicals.

In the synthetic process according to Scheme 1, firstly a phenyl-naphthyl compound is prepared by Suzuki coupling of a phenyl derivative to a naphthyl derivative which carries a carboxylate group, the precursor for the bridging methylene group of the fluorene. The carboxylate group is converted, by addition of an organometallic compound, into the tertiary alcohol, which undergoes the ring-closure reaction to give the methylene bridge of the fluorene. The further steps are halogenation, coupling of an aryl group to the outer ring of the fluorene derivative, re-halogenation and subsequent Buchwald coupling. In the Buchwald coupling, in each case two fluorene derivatives are coupled to an arylamine, giving the compound according to the invention.

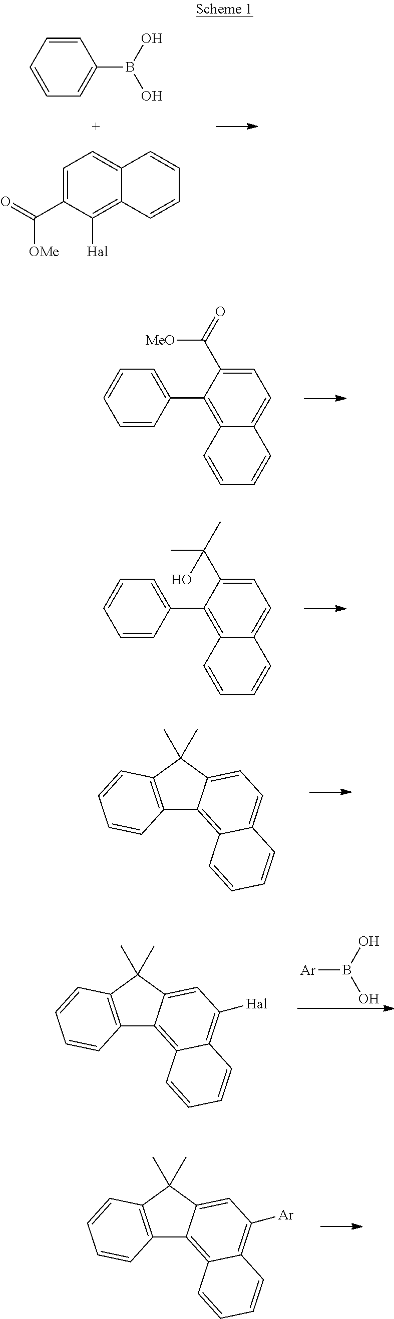

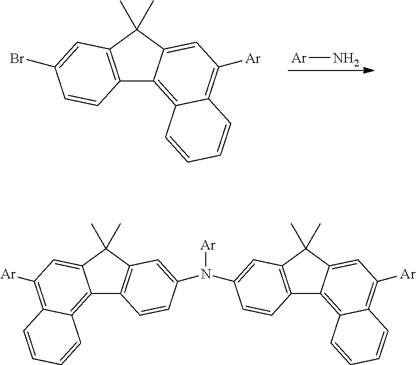

In the process according to Scheme 2, the phenyl-naphthyl compound which contains a carboxylate group as precursor of the methylene bridge of the benzofluorene is prepared via a Suzuki coupling already to the arylamino compound. Addition of an organometallic nucleophile and subsequent ring closure for the formation of a methylene bridge result in the desired compound according to the invention.

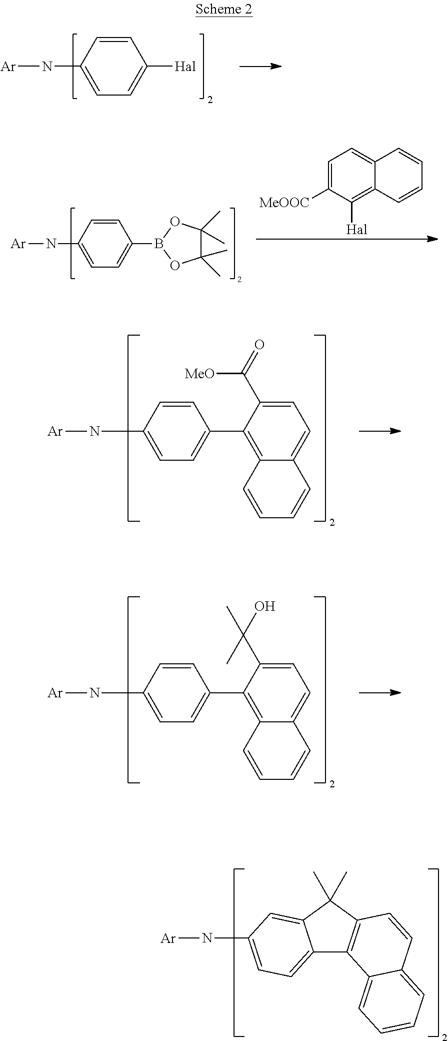

In the process according to Scheme 3, the procedure is substantially analogous to Scheme 2 with the difference that the amine employed contains three instead of two reactive phenyl groups, so that overall three benzofluorenyl groups are formed bonded to the central nitrogen atom.

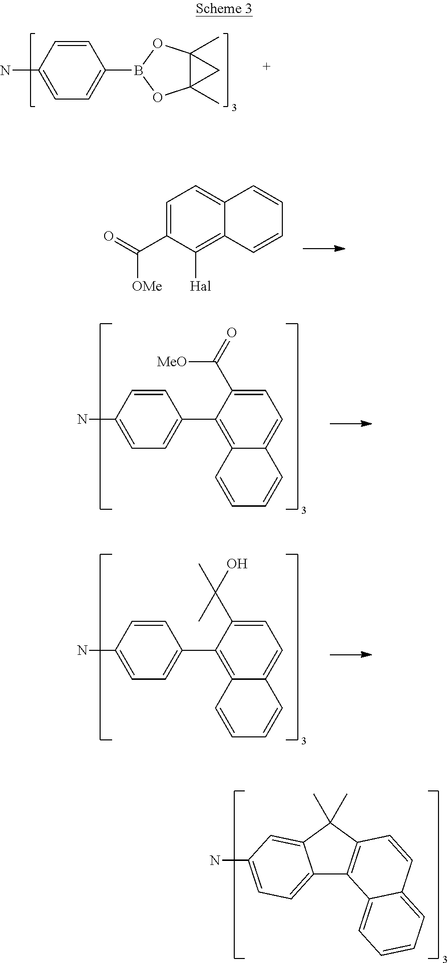

The above-mentioned synthetic processes according to Schemes 1 to 3 may be followed by further functionalisation reactions in which the compounds according to the invention obtained are reacted further.

The compounds according to the invention described above, in particular compounds which are substituted by reactive leaving groups, such as bromine, iodine, chlorine, boronic acid or boronic acid ester, can be used as monomers for the production of corresponding oligomers, dendrimers or polymers. Suitable reactive leaving groups are, for example, bromine, iodine, chlorine, boronic acids, boronic acid esters, amines, alkenyl or alkynyl groups having a terminal C—C double bond or C—C triple bond, oxiranes, oxetanes, groups which undergo a cycloaddition, for example a 1,3-dipolar cycloaddition, such as, for example, dienes or azides, carboxylic acid derivatives, alcohols and silanes.

The invention therefore furthermore relates to oligomers, polymers or dendrimers containing one or more compounds of the formula (I), where the bond(s) to the polymer, oligomer or dendrimer may be localised at any desired positions in formula (I) which are substituted by R¹ or R². Depending on the linking of the compound of the formula (I), the compound is a constituent of a side chain of the oligomer or polymer or a constituent of the main chain. An oligomer in the sense of this invention is taken to mean a compound which is built up from at least three monomer units. A polymer in the sense of the invention is taken to mean a compound which is built up from at least ten monomer units. The polymers, oligomers or dendrimers according to the invention may be conjugated, partially conjugated or non-conjugated. The oligomers or polymers according to the invention may be linear, branched or dendritic. In the structures linked in a linear manner, the units of the formula (I) may be linked directly to one another or they may be linked to one another via a divalent group, for example via a substituted or unsubstituted alkylene group, via a heteroatom or via a divalent aromatic or heteroaromatic group. In branched and dendritic structures, for example, three or more units of the formula (I) may be linked via a trivalent or polyvalent group, for example via a trivalent or polyvalent aromatic or heteroaromatic group, to form a branched or dendritic oligomer or polymer.

The same preferences as described above for compounds of the formula (I) apply to the recurring units of the formula (I) in oligomers, dendrimers and polymers.

For the preparation of the oligomers or polymers, the monomers according to the invention are homopolymerised or copolymerised with further monomers. Suitable and preferred comonomers are selected from fluorenes (for example in accordance with EP 842208 or WO 2000/22026), spirobifluorenes (for example in accordance with EP 707020, EP 894107 or WO 2006/061181), para-phenylenes (for example in accordance with WO 1992/18552), carbazoles (for example in accordance with WO 2004/070772 or WO 2004/113468), thiophenes (for example in accordance with EP 1028136), dihydrophenanthrenes (for example in accordance with WO 2005/014689 or WO 2007/006383), cis- and trans-indenofluorenes (for example in accordance with WO 2004/041901 or WO 2004/113412), ketones (for example in accordance with WO 2005/040302), phenanthrenes (for example in accordance with WO 2005/104264 or WO 2007/017066) or also a plurality of these units. The polymers, oligomers and dendrimers usually also contain further units, for example emitting (fluorescent or phosphorescent) units, such as, for example, vinyltriarylamines (for example in accordance with WO 2007/068325) or phosphorescent metal complexes (for example in accordance with WO 2006/003000), and/or charge-transport units, in particular those based on triarylamines.

The polymers, oligomers and dendrimers according to the invention have advantageous properties, in particular long lifetimes, high efficiencies and good colour coordinates.

The polymers and oligomers according to the invention are generally prepared by polymerisation of one or more types of monomer, at least one monomer of which results in recurring units of the formula (I) in the polymer. Suitable polymerisation reactions are known to the person skilled in the art and are described in the literature. Particularly suitable and preferred polymerisation reactions which result in C—C or C—N links are the following:

(A) SUZUKI polymerisation;

(B) YAMAMOTO polymerisation;

(C) STILLE polymerisation; and

(D) HARTWIG-BUCHWALD polymerisation.

The way in which the polymerisation can be carried out by these methods and the way in which the polymers can then be separated off from the reaction medium and purified is known to the person skilled in the art and is described in detail in the literature, for example in WO 2003/048225, WO 2004/037887 and WO 2004/037887.

The present invention thus also relates to a process for the preparation of the polymers, oligomers and dendrimers according to the invention, which is characterised in that they are prepared by SUZUKI polymerisation, YAMAMOTO polymerisation, STILLE polymerisation or HARTWIG-BUCHWALD polymerisation. The dendrimers according to the invention can be prepared by processes known to the person skilled in the art or analogously thereto. Suitable processes are described in the literature, such as, for example, in Frechet, Jean M. J.; Hawker, Craig J., “Hyperbranched polyphenylene and hyperbranched polyesters: new soluble, three-dimensional, reactive polymers”, Reactive & Functional Polymers (1995), 26(1-3), 127-36; Janssen, H. M.; Meijer, E. W., “The synthesis and characterization of dendritic molecules”, Materials Science and Technology (1999), 20 (Synthesis of Polymers), 403-458; Tomalia, Donald A., “Dendrimer molecules”, Scientific American (1995), 272(5), 62-6; WO 2002/067343 A1 and WO 2005/026144 A1.

For the processing of the compounds according to the invention from the liquid phase, for example by spin coating or by printing processes, formulations of the compounds according to the invention are necessary. These formulations can be, for example, solutions, dispersions or emulsions. It may be preferred to use mixtures of two or more solvents for this purpose. Suitable and preferred solvents are, for example, toluene, anisole, o-, m- or p-xylene, methyl benzoate, mesitylene, tetralin, veratrol, THF, methyl-THF, THP, chlorobenzene, dioxane, phenoxytoluene, in particular 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetol, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore furthermore relates to a formulation, in particular a solution, dispersion or emulsion, comprising at least one compound of the formula (I) or at least one polymer, oligomer or dendrimer containing at least one unit of the formula (I), and at least one solvent, preferably an organic solvent. The way in which solutions of this type can be prepared is known to the person skilled in the art and is described, for example, in WO 2002/072714, WO 2003/019694 and the literature cited therein.

The compounds of the formula (I) according to the invention are suitable for use in electronic devices, in particular in organic electroluminescent devices (OLEDs). Depending on the substitution, the compounds are employed in different functions and layers.

The invention therefore furthermore relates to the use of a compound of the formula (I) in an electronic device. The electronic device here is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and particularly preferably organic electroluminescent devices (OLEDs).

The invention furthermore relates to an electronic device comprising at least one compound of the formula (I). The electronic device is preferably selected from the devices indicated above. Particular preference is given to an organic electroluminescent device comprising anode, cathode and at least one emitting layer, characterised in that at least one organic layer comprises at least one compound of the formula (I).

Apart from cathode, anode and emitting layer, the organic electroluminescent device may also comprise further layers. These are selected, for example, from in each case one or more hole-injection layers, hole-transport layers, hole-blocking layers, electron-transport layers, electron-injection layers, electron-blocking layers, exciton-blocking layers, interlayers, charge-generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer) and/or organic or inorganic p/n junctions. However, it should be pointed out that each of these layers does not necessarily have to be present and the choice of layers is always dependent on the compounds used and in particular also on whether the electroluminescent device is fluorescent or phosphorescent.

The sequence of the layers of the organic electroluminescent device is preferably the following:

anode-hole-injection layer-hole-transport layer-emitting layer-electron-transport layer-electron-injection layer-cathode.

It should again be pointed out here that not all the said layers have to be present, and/or that further layers may additionally be present.

The organic electroluminescent device according to the invention may comprise a plurality of emitting layers. These emission layers in this case particularly preferably have in total a plurality of emission maxima between 380 nm and 750 nm, resulting overall in white emission, i.e. various emitting compounds which are able to fluoresce or phosphoresce and which emit blue or yellow or orange or red light are used in the emitting layers. Particular preference is given to three-layer systems, i.e. systems having three emitting layers, where at least one of these layers preferably comprises at least one compound of the formula (I) and where the three layers exhibit blue, green and orange or red emission (for the basic structure see, for example, WO 2005/011013). It should be noted that, for the generation of white light, an emitter compound used individually which emits in a broad wavelength range may also be suitable instead of a plurality of emitter compounds emitting in colour.

The compounds according to the invention may alternatively and/or additionally also be present in the hole-transport layer or in another layer.

It is preferred for the compound of the formula (I) to be employed in an emitting layer. In particular, the compound of the formula (I) is suitable for use as emitting material (emitter compound).

The compound according to the invention is particularly suitable for use as blue-emitting emitter compound. The electronic device concerned may comprise a single emitting layer comprising the compound according to the invention or it may comprise two or more emitting layers. The further emitting layers here may comprise one or more compounds according to the invention or alternatively other compounds.

If the compound according to the invention is employed as emitting material in an emitting layer, it is preferably employed in combination with one or more matrix materials.

The proportion of the compound according to the invention in the mixture of the emitting layer is in this case preferably between 0.1 and 50.0% by vol., particulary preferably between 0.5 and 20.0% by vol., very particularly preferably between 1.0 and 10.0% by vol. Correspondingly, the proportion of the matrix material or matrix materials is between 50.0 and 99.9% by vol., particularly preferably between 80.0 and 99.5% by vol., very particularly preferably between 90.0 and 99.0% by vol.

Preferred matrix materials for use in combination with the materials according to the invention as emitters are selected from the classes of the oligoarylenes (for example 2,2′,7,7′-tetraphenylspirobifluorene in accordance with EP 676461 or dinaphthylanthracene), in particular the oligoarylenes containing condensed aromatic groups, the oligoarylenevinylenes (for example DPVBi or spiro-DPVBi in accordance with EP 676461), the polypodal metal complexes (for example in accordance with WO 2004/081017), the hole-conducting compounds (for example in accordance with WO 2004/058911), the electron-conducting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (for example in accordance with WO 2008/145239). Particularly preferred matrix materials are selected from the classes of the oligoarylenes, comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes, comprising anthracene, benzanthracene, benzophenanthrene and/or pyrene or atropisomers of these compounds. An oligoarylene in the sense of this invention is intended to be taken to mean a compound in which at least three aryl or arylene groups are bonded to one another.

Preferred matrix materials for use in combination with the compound of the formula (I) in the emitting layer are depicted in the following table.

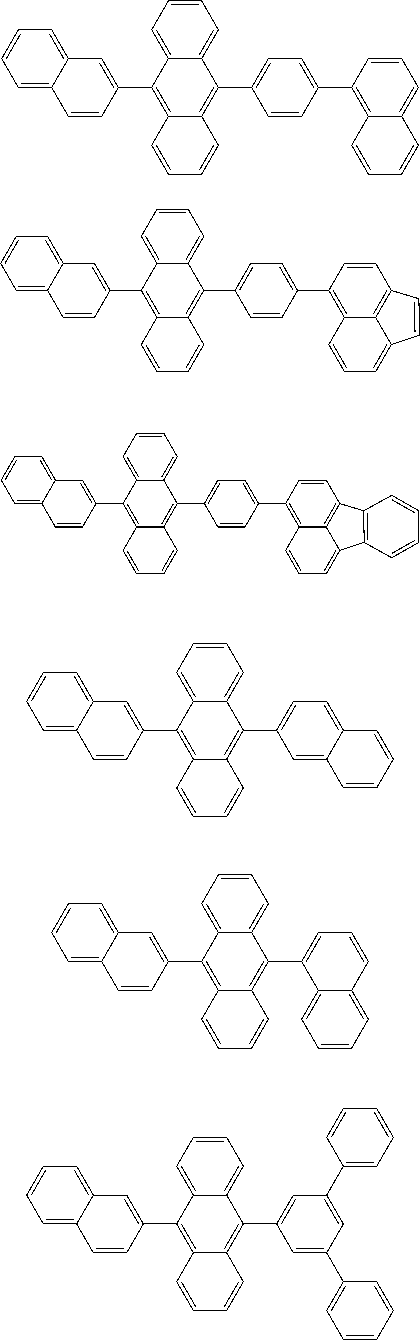

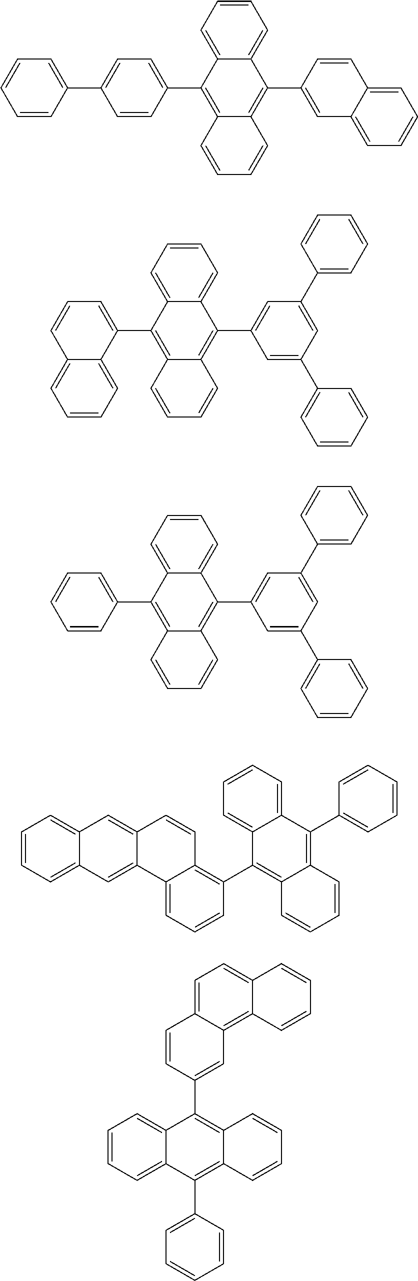

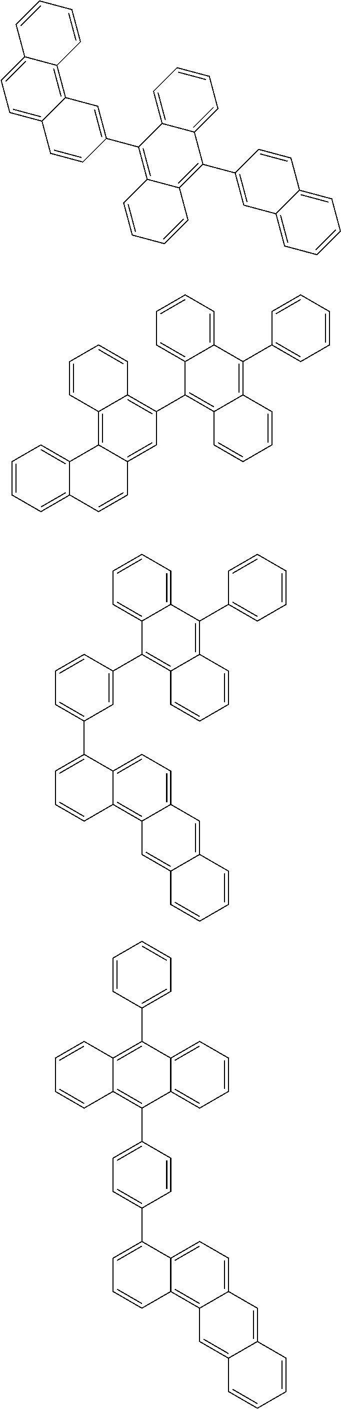

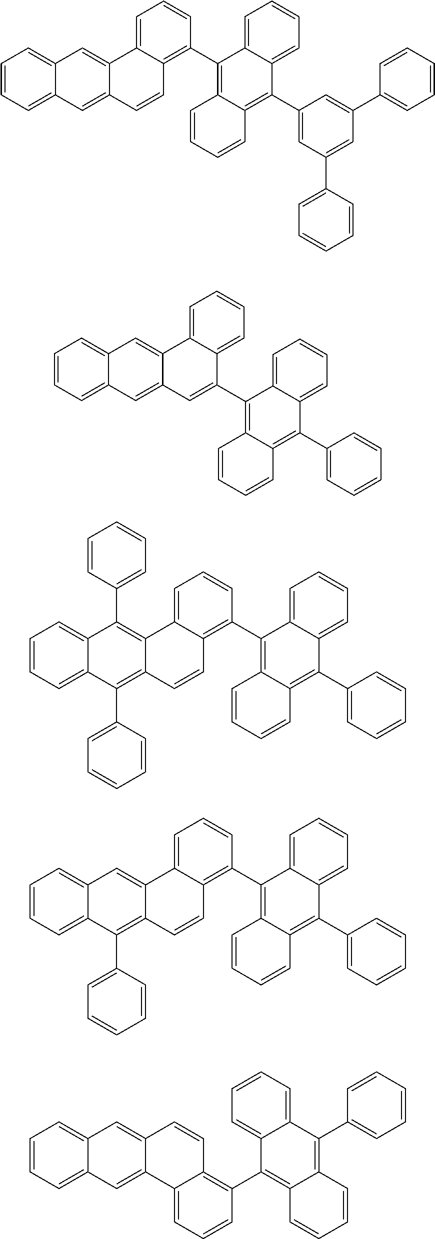

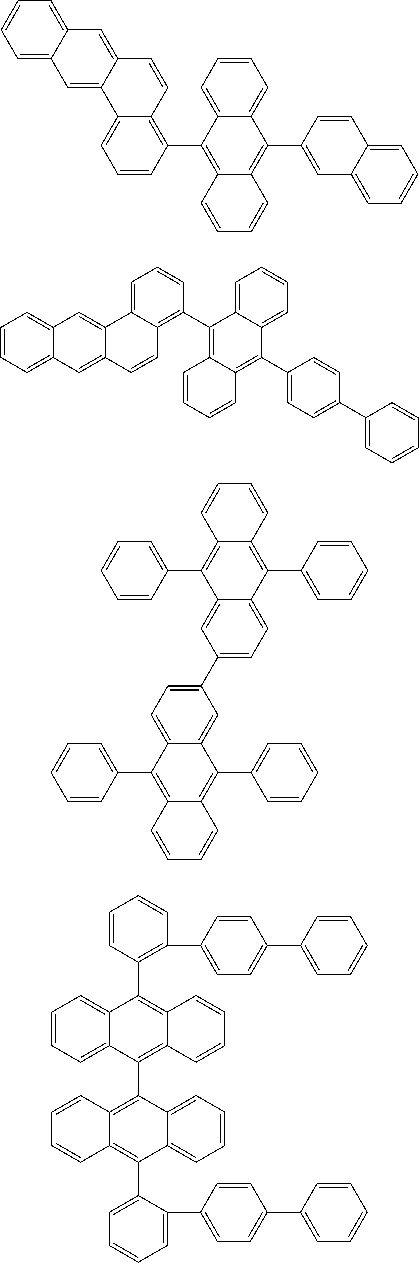

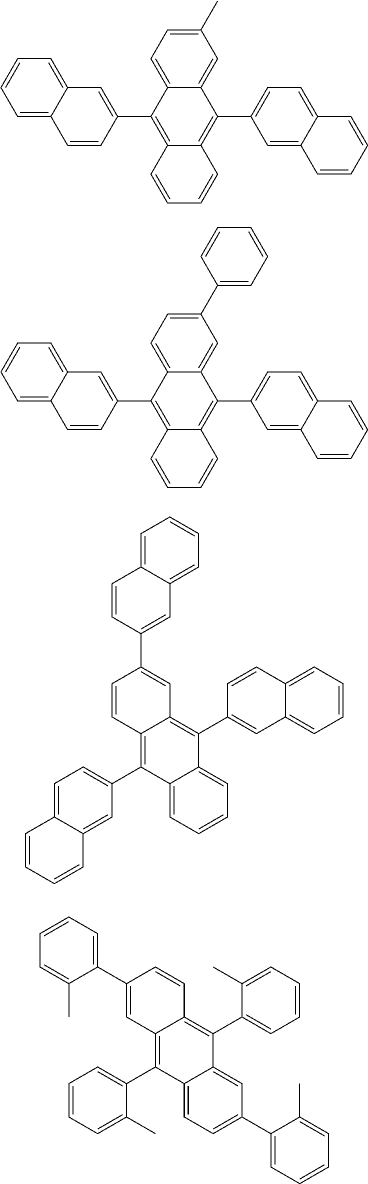

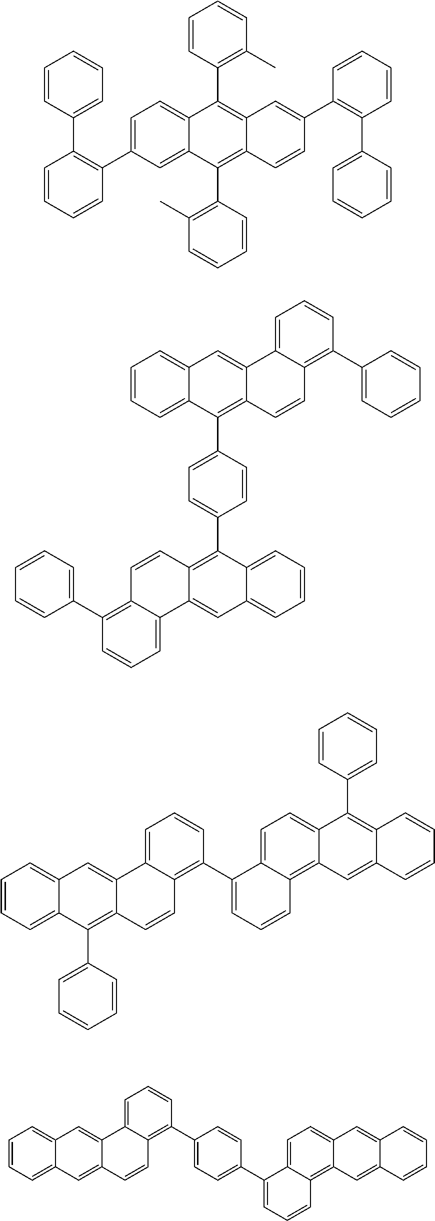

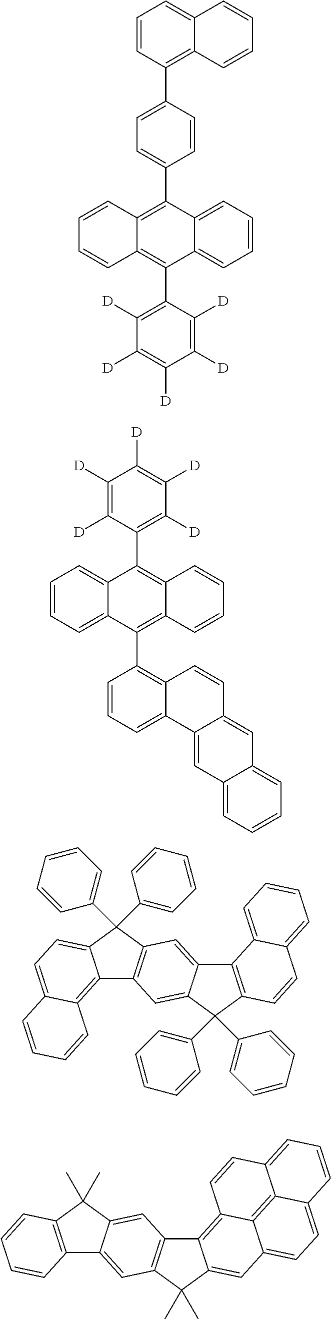

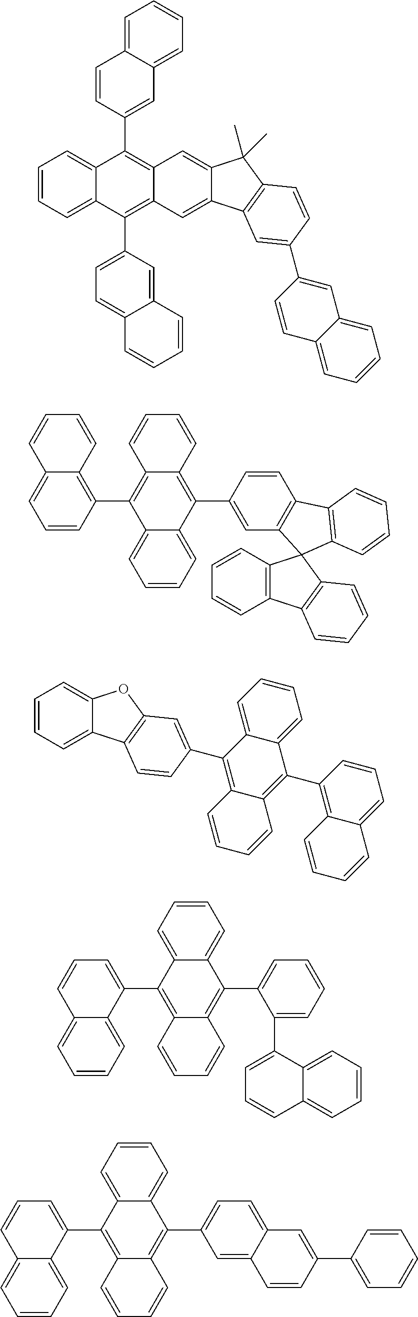

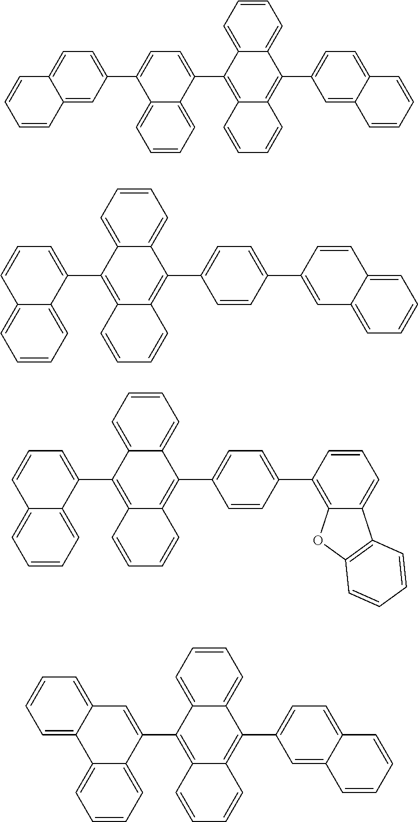

The compounds according to the invention can also be employed in other layers, for example as hole-transport materials in a hole-injection or hole-transport layer or electron-blocking layer or as matrix materials in an emitting layer, preferably as matrix materials for phosphorescent emitters.

If the compound of the formula (I) is employed as hole-transport material in a hole-transport layer, a hole-injection layer or an electron-blocking layer, the compound can be employed as pure material, i.e. in a proportion of 100%, in the hole-transport layer, or it can be employed in combination with one or more further compounds. According to a preferred embodiment, the organic layer comprising the compound of the formula (I) then additionally comprises one or more p-dopants. The p-dopants employed in accordance with the present invention are preferably organic electron-acceptor compounds which are able to oxidise one or more of the other compounds of the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. No. 8,044,390, U.S. Pat. No. 8,057,712, WO 2009/003455, WO 2010/094378, WO 2011/120709, US 2010/0096600 and WO 2012/095143.

If the compound of the formula (I) is employed as matrix material in combination with a phosphorescent emitter in an emitting layer, the phosphorescent emitter is preferably selected from the classes and embodiments of phosphorescent emitters indicated below. Furthermore, one or more further matrix materials are preferably present in the emitting layer in this case.

So-called mixed-matrix systems of this type preferably comprise two or three different matrix materials, particularly preferably two different matrix materials. It is preferred here for one of the two materials to be a material having hole-transporting properties and for the other material to be a material having electron-transporting properties. The compound of the formula (I) is preferably the material having hole-transporting properties.

However, the desired electron-transporting and hole-transporting properties of the mixed-matrix components may also be combined mainly or completely in a single mixed-matrix component, where the further mixed-matrix component or components satisfy other functions. The two different matrix materials may be present here in a ratio of 1:50 to 1:1, preferably 1:20 to 1:1, particularly preferably 1:10 to 1:1 and very particularly preferably 1:4 to 1:1. Mixed-matrix systems are preferably employed in phosphorescent organic electroluminescent devices. Further details on mixed-matrix systems are contained, inter alia, in the application WO 2010/108579.

Particularly suitable matrix materials which can be used as matrix components of a mixed-matrix system in combination with the compounds according to the invention are selected from the preferred matrix materials for phosphorescent emitters indicated below or the preferred matrix materials for fluorescent emitters, depending on what type of emitter compound is employed in the mixed-matrix system.

Generally preferred classes of material for use as corresponding functional materials in the organic electroluminescent devices according to the invention are indicated below.

Suitable phosphorescent emitters are, in particular, compounds which emit light, preferably in the visible region, on suitable excitation and in addition contain at least one atom having an atomic number greater than 20, preferably greater than 38 and less than 84, particularly preferably greater than 56 and less than 80. The phosphorescent emitters used are preferably compounds which contain copper, molybdenum, tungsten, rhenium, ruthenium, osmium, rhodium, iridium, palladium, platinum, silver, gold or europium, in particular compounds which contain iridium, platinum or copper.

For the purposes of the present invention, all luminescent iridium, platinum or copper complexes are regarded as phosphorescent compounds.

Examples of the phosphorescent emitters described above are revealed by the applications WO 2000/70655, WO 2001/41512, WO 2002/02714, WO 2002/15645, EP 1191613, EP 1191612, EP 1191614, WO 2005/033244, WO 2005/019373 and US 2005/0258742. In general, all phosphorescent complexes as used in accordance with the prior art for phosphorescent OLEDs and as are known to the person skilled in the art in the area of organic electroluminescent devices are suitable for use in the devices according to the invention. The person skilled in the art will also be able to employ further phosphorescent complexes without inventive step in combination with the compounds according to the invention in OLEDs.

Preferred fluorescent emitters, besides the compounds according to the invention, are selected from the class of the arylamines. An arylamine or aromatic amine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, particularly preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 9,10-position. Aromatic pyrenamines, pyrenediamines, chrysenamines and chrysenediamines are defined analogously thereto, where the diarylamino groups are preferably bonded to the pyrene in the 1-position or in the 1,6-position.

Preferred matrix materials for use with fluorescent emitters compounds are indicated above.

Preferred matrix materials for phosphorescent emitters are aromatic ketones, aromatic phosphine oxides or aromatic sulfoxides or sulfones, for example in accordance with WO 2004/013080, WO 2004/093207, WO 2006/005627 or WO 2010/006680, triarylamines, carbazole derivatives, for example CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example in accordance with WO 2007/063754 or WO 2008/056746, indenocarbazole derivatives, for example in accordance with WO 2010/136109, WO 2011/000455 or WO 2013/041176, azacarbazole derivatives, for example in accordance with EP 1617710, EP 1617711, EP 1731584, JP 2005/347160, bipolar matrix materials, for example in accordance with WO 2007/137725, silanes, for example in accordance with WO 2005/111172, azaboroles or boronic esters, for example in accordance with WO 2006/117052, triazine derivatives, for example in accordance with WO 2010/015306, WO 2007/063754 or WO 2008/056746, zinc complexes, for example in accordance with EP 652273 or WO 2009/062578, diazasilole or tetraazasilole derivatives, for example in accordance with WO 2010/054729, diazaphosphole derivatives, for example in accordance with WO 2010/054730, bridged carbazole derivatives, for example in accordance with US 2009/0136779, WO 2010/050778, WO 2011/042107, WO 2011/088877 or WO 2012/143080, triphenylene derivatives, for example in accordance with WO 2012/048781, or lactams, for example in accordance with WO 2011/116865 or WO 2011/137951.

Besides the compounds according to the invention, suitable charge-transport materials, as can be used in the hole-injection or hole-transport layer or electron-blocking layer or in the electron-transport layer of the electronic device according to the invention, are, for example, the compounds disclosed in Y. Shirota et al., Chem. Rev. 2007, 107(4), 953-1010, or other materials as are employed in these layers in accordance with the prior art.

Materials which can be used for the electron-transport layer are all materials as are used in accordance with the prior art as electron-transport materials in the electron-transport layer. Particularly suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Furthermore suitable materials are derivatives of the above-mentioned compounds, as disclosed in JP 2000/053957, WO 2003/060956, WO 2004/028217, WO 2004/080975 and WO 2010/072300.

Preferred hole-transport materials which can be used in a hole-transport, hole-injection or electron-blocking layer in the electroluminescent device according to the invention are indenofluorenamine derivatives (for example in accordance with WO 06/122630 or WO 06/100896), the amine derivatives disclosed in EP 1661888, hexaazatriphenylene derivatives (for example in accordance with WO 01/049806), amine derivatives containing condensed aromatic rings (for example in accordance with U.S. Pat. No. 5,061,569), the amine derivatives disclosed in WO 95/09147, monobenzoindenofluorenamines (for example in accordance with WO 08/006449), dibenzoindenofluorenamines (for example in accordance with WO 07/140847), spirobifluorenamines (for example in accordance with WO 2012/034627 or WO 2013/120577), fluorenamines (for example in accordance with the as yet unpublished applications EP 12005369.9, EP 12005370.7 and EP 12005371.5), spirodibenzopyranamines (for example in accordance with WO 2013/083216) and dihydroacridine derivatives (for example in accordance with WO 2012/150001). The compounds according to the invention can also be used as hole-transport materials.

The cathode of the organic electroluminescent device preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). Also suitable are alloys comprising an alkali metal or alkaline-earth metal and silver, for example an alloy comprising magnesium and silver. In the case of multilayered structures, further metals which have a relatively high work function, such as, for example, Ag or Al, can also be used in addition to the said metals, in which case combinations of the metals, such as, for example, Ca/Ag, Mg/Ag or Ag/Ag, are generally used. It may also be preferred to introduce a thin interlayer of a material having a high dielectric constant between a metallic cathode and the organic semiconductor. Suitable for this purpose are, for example, alkali metal fluorides or alkaline-earth metal fluorides, but also the corresponding oxides or carbonates (for example LiF, Li₂O, BaF₂, MgO, NaF, CsF, Cs₂CO₃, etc.). Furthermore, lithium quinolinate (LiQ) can be used for this purpose. The layer thickness of this layer is preferably between 0.5 and 5 nm.

The anode preferably comprises materials having a high work function. The anode preferably has a work function of greater than 4.5 eV vs. vacuum. Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent or partially transparent in order to facilitate either irradiation of the organic material (organic solar cells) or the coupling-out of light (OLEDs, O-lasers). Preferred anode materials here are conductive mixed metal oxides. Particular preference is given to indium tin oxide (ITO) or indium zinc oxide (IZO). Preference is furthermore given to conductive, doped organic materials, in particular conductive doped polymers.

The device is appropriately (depending on the application) structured, provided with contacts and finally sealed, since the lifetime of the devices according to the invention is shortened in the presence of water and/or air.

In a preferred embodiment, the organic electroluminescent device according to the invention is characterised in that one or more layers are coated by means of a sublimation process, in which the materials are applied by vapour deposition in vacuum sublimation units at an initial pressure of less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are coated by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and are thus structured (for example M. S. Arnold et al., Appl. Phys. Left. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, but particularly preferably LITI (light induced thermal imaging, thermal transfer printing) or ink-jet printing. Soluble compounds of the formula (I) are necessary for this purpose. High solubility can be achieved through suitable substitution of the compounds.

For the production of an organic electroluminescent device according to the invention, it is furthermore preferred to apply one or more layers from solution and one or more layers by a sublimation process.

In accordance with the invention, the electronic devices comprising one or more compounds according to the invention can be employed in displays, as light sources in lighting applications and as light sources in medical and/or cosmetic applications (for example light therapy).

### WORKING EXAMPLES

### A) Synthesis Examples

### A-1) Variant I

### General Reaction Scheme:

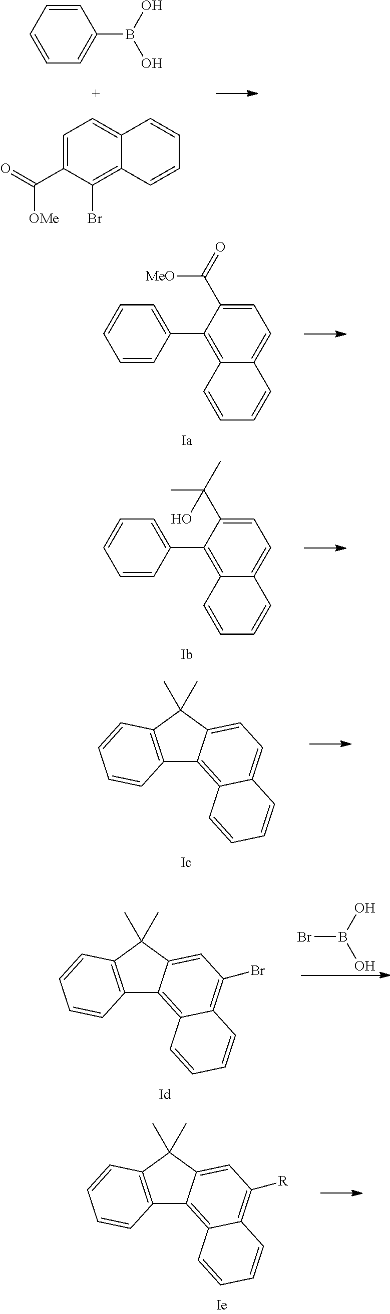

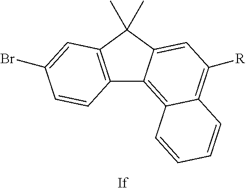

### A-1-1) Synthesis of Building Blocks (If-1) to (If-5)

### Methyl 1-phenylnaphthalene-2-carboxylate (Ia)

Methyl 1-bromonaphthalene-2-carboxylate (70.0 g, 264 mmol), phenylboronic acid (38.6 g, 317 mmol) and potassium phosphate monohydrate 182 g, 792 mmol) are dissolved in a mixture of 0.2 l of toluene, 0.2 l of dioxane and 0.2 l of water, and palladium acetate (1.18 g, 5.3 mmol) and tri-ortho-tolylphosphine (3.2 g, 10.6 mmol) are added. The batch is heated under reflux overnight, cooled to room temperature and extended with dist. water. After phase separation, the aqueous phase is extracted a number of times with toluene. The combined organic phases are washed with dist. water, dried over magnesium sulfate and filtered through aluminium oxide. The organic phase is evaporated to give an orange oil, giving 69 g of product (99% of theory).

### 2-(1-Phenylnaphthalen-2-yl)propan-2-ol (Ib)

(Ia) (69 g, 264 mmol) and cerium(III) chloride (71 g, 291 mmol) are dissolved in 500 ml of THF, and methylmagnesium chloride (3 M solution in THF) (308 ml, 925 mmol) is added at 0° C. The reaction solution is allowed to warm to room temperature overnight in an ice bath. The batch is carefully hydrolysed using saturated NH₄Cl solution and neutralised using 4% hydrochloric acid. The mixture is extended with dist. water and extracted thoroughly with toluene. The combined organic phases are washed a number of times with dist. water and once with sodium hydrogencarbonate solution and dried over magnesium sulfate. Removal of the solvent in vacuo gives a pale-beige solid, which is recrystallised from a heptane/toluene mixture, giving 52 g (75% of theory) as a colourless solid.

### 7,7-Dimethyl-7H-benzo[c]fluorene (Ic)

(Ib) (52 g, 198 mmol) is dissolved in 500 ml of DCM, and methanesulfonic acid (64 ml, 991 mmol) and polyphosphoric acid (77 g, 793 mmol) are added at 0° C. The reaction solution is allowed to warm to room temperature overnight. The mixture is extended with ethanol and evaporated. The residue is dissolved in toluene, washed with NaOH solution and dist. water and dried over magnesium sulfate. After removal of the solvent in vacuo, the solid is recrystallised from ethanol, giving 44 g of a yellow solid (91% of theory).

### 5-Bromo-7,7-dimethyl-7H-benzo[c]fluorene (Id)

(Ic) (38.2 g, 156 mmol) is dissolved in 0.3 l of chloroform and cooled to 0° C. A dibromine solution (117 g, 660 mmol) in 0.2 l of chloroform is added dropwise with stirring at such a rate that the reaction temperature does not rise above 5° C. The reaction is warmed to room temperature overnight in an ice bath. 200 ml of a 10% sodium thiosulfate solution are added, and the phases are separated. The aqueous phase is extracted a number of times with DCM. The organic phase is washed with dist. water, dried and freed from the solvent. The solid obtained is recrystallised from toluene/heptane, giving 50 g of a colourless solid (99% of theory).

### 7,7-Dimethyl-5-phenyl-7H-benzo[c]fluorene (Ie-1)

(Id) (28.5 g, 88 mmol), phenylboronic acid (13.2 g, 106 mmol) and potassium carbonate (30.5 g, 220 mmol) are dissolved in a mixture of 150 ml of toluene and 150 ml of water, and tetrakis(triphenylphosphine)palladium (1.02 g, 0.9 mmol) is added. The batch is heated under reflux overnight, cooled to room temperature and extended with dist. water. After phase separation, the aqueous phase is extracted a number of times with toluene. The combined organic phases are washed with dist. water, dried over magnesium sulfate and filtered through AlOx and silica gel. The organic phase is evaporated, and the resultant solid is washed with ethanol, giving 25.9 g (92% of theory) of product.

The following compounds are prepared analogously:

| | Bromoarene | R | Product | Yield |
|---|---|---|---|---|
| (Ie-1) | 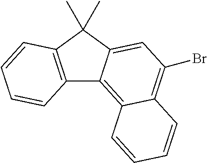 [SMILES: CC1(C)c2ccccc2-c2c1cc(Br)c1ccccc21] | 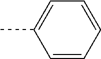 | 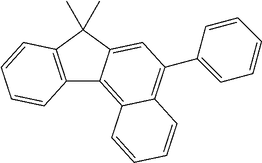 [SMILES: CC1(C)c2ccccc2-c2c1cc(-c1ccccc1)c1ccccc21] | 92% |
| (Ie-2) | 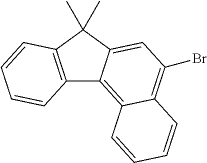 [SMILES: CC1(C)c2ccccc2-c2c1cc(Br)c1ccccc21] | 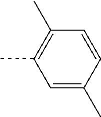 | 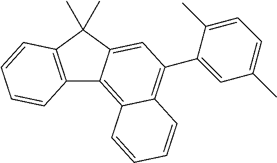 [SMILES: Cc1ccc(C)c(-c2cc3c(c4ccccc24)-c2ccccc2C3(C)C)c1] | 89% |
| (Ie-3) | 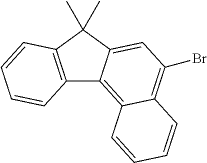 [SMILES: CC1(C)c2ccccc2-c2c1cc(Br)c1ccccc21] | 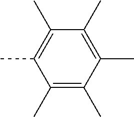 | 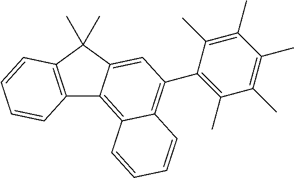 [SMILES: Cc1c(C)c(C)c(-c2cc3c(c4ccccc24)-c2ccccc2C3(C)C)c(C)c1C] | 82% |
| (Ie-4) | 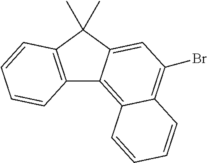 [SMILES: CC1(C)c2ccccc2-c2c1cc(Br)c1ccccc21] | 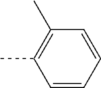 | 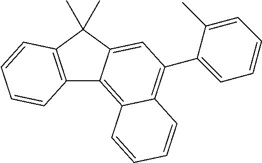 [SMILES: Cc1ccccc1-c1cc2c(c3ccccc13)-c1ccccc1C2(C)C] | 91% |
| (Ie-5) | 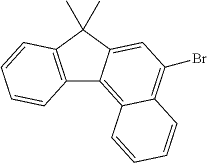 [SMILES: CC1(C)c2ccccc2-c2c1cc(Br)c1ccccc21] | 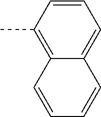 | 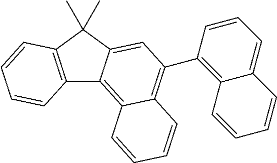 [SMILES: CC1(C)c2ccccc2-c2c1cc(-c1cccc3ccccc13)c1ccccc21] | 80% |
| (Ie-8) | 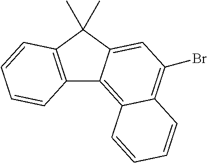 [SMILES: CC1(C)c2ccccc2-c2c1cc(Br)c1ccccc21] | 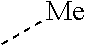 | 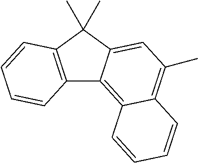 [SMILES: Cc1cc2c(c3ccccc13)-c1ccccc1C2(C)C] | 85% |
| (Ie-9) | 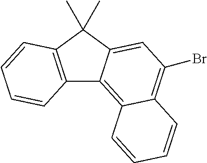 [SMILES: CC1(C)c2ccccc2-c2c1cc(Br)c1ccccc21] | 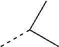 | 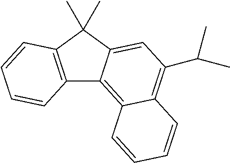 [SMILES: CC(C)c1cc2c(c3ccccc13)-c1ccccc1C2(C)C] | 78% |
| (Ie-10) | 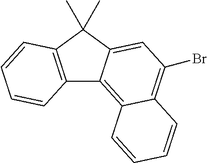 [SMILES: CC1(C)c2ccccc2-c2c1cc(Br)c1ccccc21] | 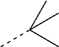 | 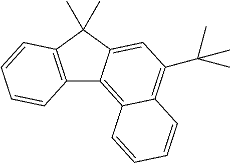 [SMILES: CC(C)(C)c1cc2c(c3ccccc13)-c1ccccc1C2(C)C] | 65% |
| (Ie-11) | 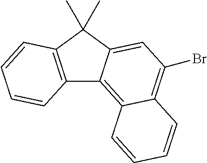 [SMILES: CC1(C)c2ccccc2-c2c1cc(Br)c1ccccc21] | 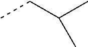 | 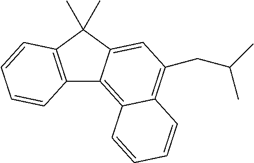 [SMILES: CC(C)Cc1cc2c(c3ccccc13)-c1ccccc1C2(C)C] | 82% |
| (Ie-12) | 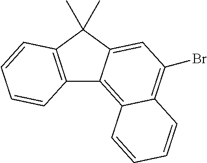 [SMILES: CC1(C)c2ccccc2-c2c1cc(Br)c1ccccc21] | 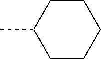 | 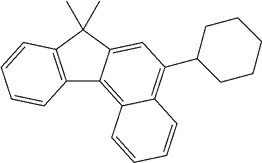 [SMILES: CC1(C)c2ccccc2-c2c1cc(C1CCCCC1)c1ccccc21] | 72% |

### 9-Bromo-7,7-dimethyl-6-phenyl-7H-benzo[c]fluorene (If-1)

(Ie-1) (25.8 g, 81 mmol) is dissolved in 0.15 l of chloroform and cooled to 0° C. A dibromine solution (13.6 g, 85 mmol) in 0.1 l of chloroform is added dropwise with stirring at such a rate that the reaction temperature does not rise above 5° C. The reaction is warmed to room temperature overnight in an ice bath. 100 ml of a 10% sodium thiosulfate solution are added, and the phases are separated. The aqueous phase is extracted a number of times with DCM. The organic phase is washed with dist. water, dried and freed from the solvent. The solid obtained is recrystallised from toluene/heptane, giving 22 g of a colourless solid (62% of theory).

The following compounds are prepared analogously:

| | Starting material | Product | Yield |
|---|---|---|---|
| (If-1) | 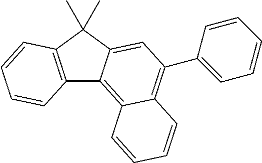 [SMILES: CC1(C)c2ccccc2-c2c1cc(-c1ccccc1)c1ccccc21] | 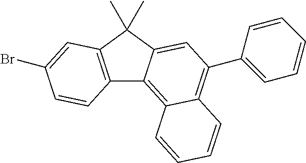 [SMILES: CC1(C)c2cc(Br)ccc2-c2c1cc(-c1ccccc1)c1ccccc21] | 62% |
| (If-2) | 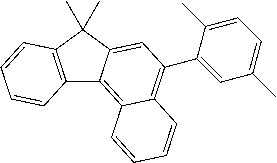 [SMILES: Cc1ccc(C)c(-c2cc3c(c4ccccc24)-c2ccccc2C3(C)C)c1] | 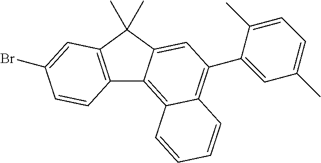 [SMILES: Cc1ccc(C)c(-c2cc3c(c4ccccc24)-c2ccc(Br)cc2C3(C)C)c1] | 75% |
| (If-3) | 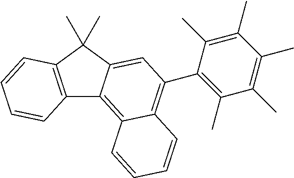 [SMILES: Cc1c(C)c(C)c(-c2cc3c(c4ccccc24)-c2ccccc2C3(C)C)c(C)c1C] | 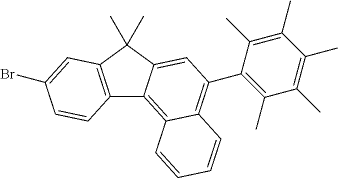 [SMILES: Cc1c(C)c(C)c(-c2cc3c(c4ccccc24)-c2ccc(Br)cc2C3(C)C)c(C)c1C] | 80% |
| (If-4) | 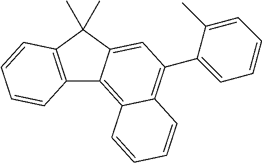 [SMILES: Cc1ccccc1-c1cc2c(c3ccccc13)-c1ccccc1C2(C)C] | 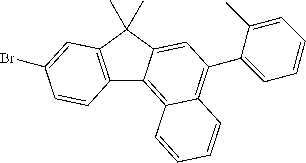 [SMILES: Cc1ccccc1-c1cc2c(c3ccccc13)-c1ccc(Br)cc1C2(C)C] | 78% |
| (lf-5) | 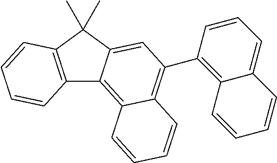 [SMILES: CC1(C)c2ccccc2-c2c1cc(-c1cccc3ccccc13)c1ccccc21] | 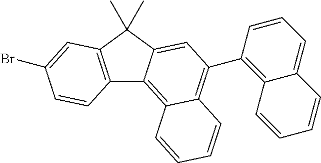 [SMILES: CC1(C)c2cc(Br)ccc2-c2c1cc(-c1cccc3ccccc13)c1ccccc21] | 77% |
| (If-8) | 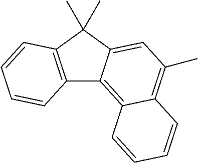 [SMILES: Cc1cc2c(c3ccccc13)-c1ccccc1C2(C)C] | 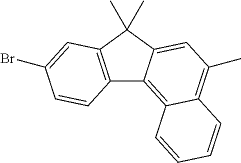 [SMILES: Cc1cc2c(c3ccccc13)-c1ccc(Br)cc1C2(C)C] | 80% |
| (If-9) | 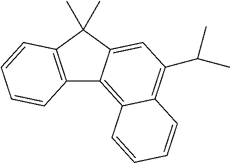 [SMILES: CC(C)c1cc2c(c3ccccc13)-c1ccccc1C2(C)C] | 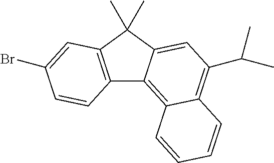 [SMILES: CC(C)c1cc2c(c3ccccc13)-c1ccc(Br)cc1C2(C)C] | 75% |
| (If-10) | 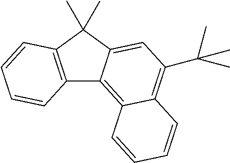 [SMILES: CC(C)(C)c1cc2c(c3ccccc13)-c1ccccc1C2(C)C] | 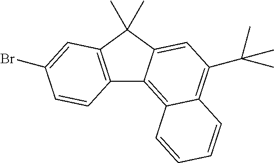 [SMILES: CC(C)(C)c1cc2c(c3ccccc13)-c1ccc(Br)cc1C2(C)C] | 83% |
| (If-11) | 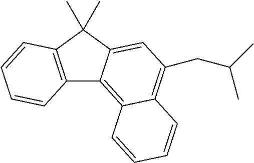 [SMILES: CC(C)Cc1cc2c(c3ccccc13)-c1ccccc1C2(C)C] | 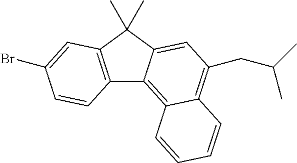 [SMILES: CC(C)Cc1cc2c(c3ccccc13)-c1ccc(Br)cc1C2(C)C] | 85% |
| (If-12) | 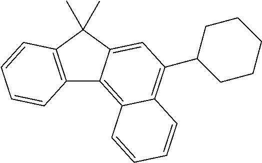 [SMILES: CC1(C)c2ccccc2-c2c1cc(C1CCCCC1)c1ccccc21] | 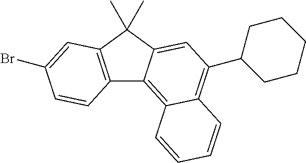 [SMILES: CC1(C)c2cc(Br)ccc2-c2c1cc(C1CCCCC1)c1ccccc21] | 89% |

### A-1-2) Synthesis of Building Block (If-6)

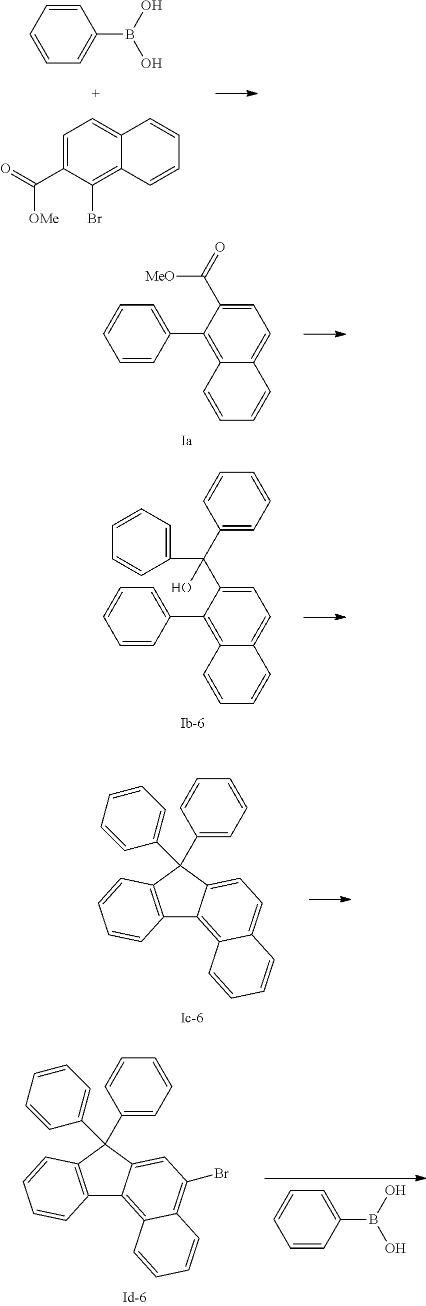

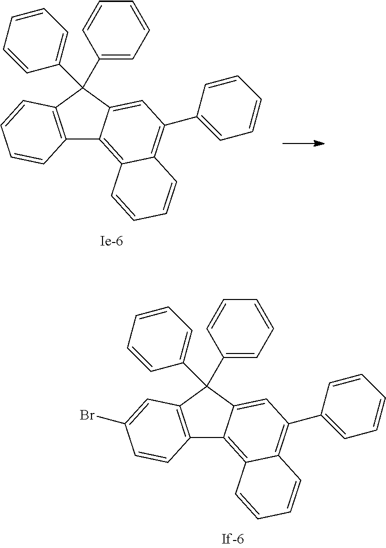

### Diphenyl-(1-phenylnaphthalen-2-yl)methanol (Ib-6)

(Ia) (35 g, 133 mmol) and cerium(III) chloride (36 g, 146 mmol) are dissolved in 250 ml of THF, and phenylmagnesium chloride (3 M solution in THF) (150 ml, 450 mmol) is added at 0° C. The reaction solution is allowed to warm to room temperature overnight in an ice bath. The batch is carefully hydrolysed using saturated NH₄Cl solution and neutralised using 4% hydrochloric acid. The mixture is extended with dist. water and extracted thoroughly with toluene. The combined organic phases are washed a number of times with dist. water and once with sodium hydrogencarbonate solution and dried over magnesium sulfate. Removal of the solvent in vacuo gives a pale-beige solid, which is recrystallised from heptane/toluene, giving 41 g (80% of theory) as a colourless solid.

Building block (Ic-6) is synthesised analogously to (Ic), with a yield of 88%.

Building block (Id-6) is synthesised analogously to (Id), with a yield of 95%.

Building block (Ie-6) is synthesised analogously to (Ie-1), with a yield of 82%.

Building block (If-6) is synthesised analogously to (If-1), with a yield of 68%.

### A-1-3) Synthesis of Building Block (Id-7)

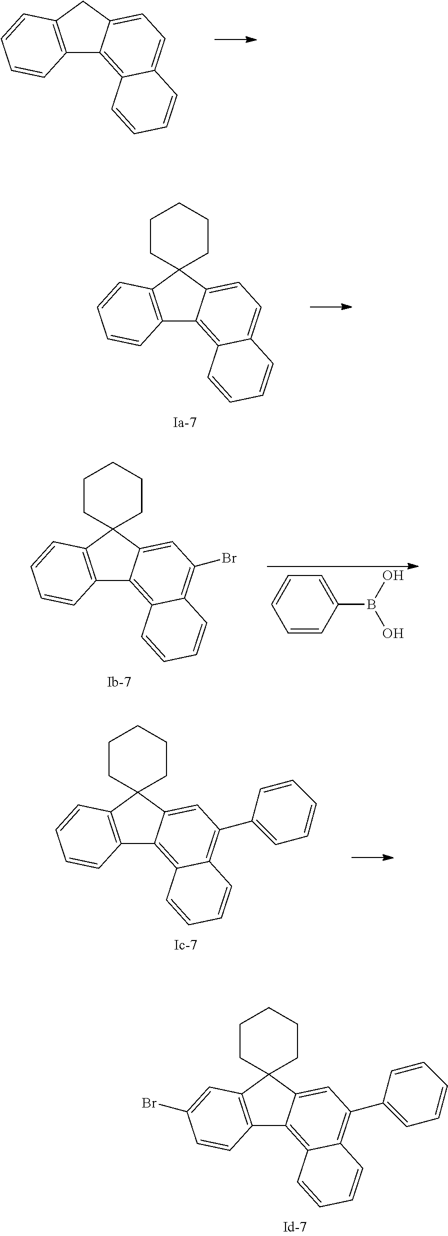

7H-Benzo[c]fluorene was synthesised in accordance with the following literature procedure: Organic Letters, 2009, Vol. 11, No. 20, 4588-4591.

### Synthesis of (Ia-7)

7H-Benzo[c]fluorene (38 g, 176 mmol), 1,5-dibromopentane (40.5 g, 176 mmol) and tetrabutylammonium bromide (32.3 g, 100 mmol) are dissolved in 0.5 l of toluene, 0.5 l of 3M NaOH solution is added, and the reaction is boiled under reflux overnight. The reaction is cooled to room temperature, the phases are separated, the aqueous phase is extracted three times with toluene. The organic phase is washed with dist. water, dried and freed from the solvent. The solid obtained is recrystallised from toluene/heptane, giving 31 g of a colourless solid (62% of theory).

Building block (Ib-7) is synthesised analogously to (Id), with a yield of 87%.

Building block (Ic-7) is synthesised analogously to (Ie-1), with a yield of 89%.

Building block (Id-7) is synthesised analogously to (If-1), with a yield of 65%.

### A-1-4) Synthesis of Target Compounds (I-1) to (I-13)

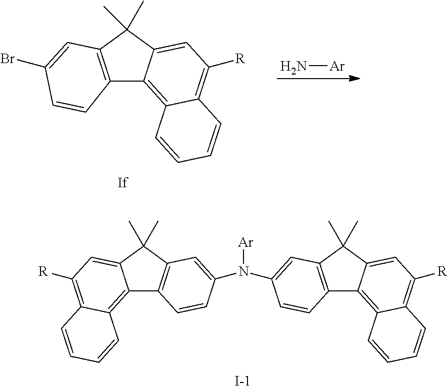

### Bis-(7,7-dimethyl-6-phenyl-7H-benzo[c]fluoren-9-yl)-p-tolylamine (I-1)

(If-1) (14 g, 35 mmol) and p-tolylamine (1.7 g, 16 mmol) are dissolved in 200 ml of toluene. 1M tri-tert-butylphosphine solution in toluene (3.6 ml, 3.6 mmol), palladium(II) acetate (5.0 g, 17.4 mmol) and sodium tert-butoxide (10 g, 104 mmol) are subsequently added to the reaction solution, which is then heated under reflux overnight. The mixture is extended with toluene and dist. water at room temperature, the organic phase is separated off, and the aqueous phase is extracted a number of times with toluene. The org. phase is dried using magnesium sulfate, filtered through AlOx and silica gel and evaporated. The residue is recrystallised from heptane/toluene, giving compound (I-1) as a pale-yellow solid (6 g, 23% of theory).

The following compounds are prepared analogously.

| Ex- | Benzo- | | | |
|---|---|---|---|---|
| ample | fluorene | | | |
| com- | deriva- | | | |
| pound | tive | Ar—NH₂ | Product | Yield |
| I-1 | If-1 | 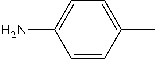 | 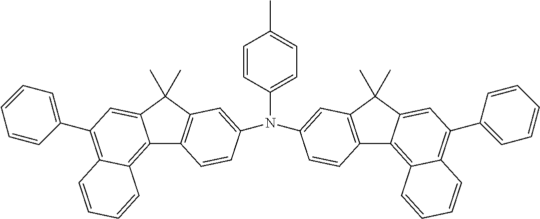 [SMILES: Cc1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)cc1] | 23% |
| I-2 | If-1 | 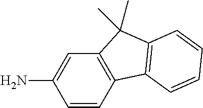 [SMILES: CC1(C)c2ccccc2-c2ccc(N)cc21] | 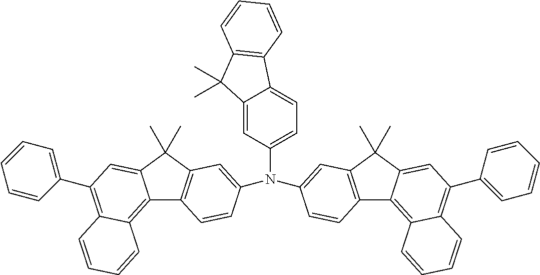 [SMILES: CC1(C)c2ccccc2-c2ccc(N(c3ccc4c(c3)C(C)(C)c3cc(-c5ccccc5)c5ccccc5c3-4)c3ccc4c(c3)C(C)(C)c3cc(-c5ccccc5)c5ccccc5c3-4)cc21] | 20% |
| I-3 | If-1 | 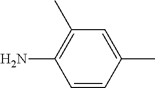 | 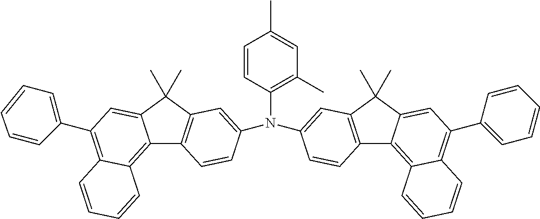 [SMILES: Cc1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)c(C)c1] | 25% |
| I-4 | If-1 | 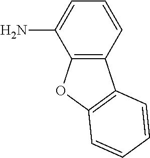 [SMILES: Nc1cccc2c1oc1ccccc12] | 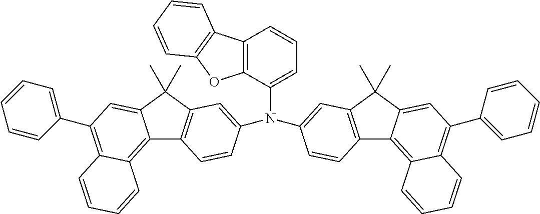 [SMILES: CC1(C)c2cc(N(c3ccc4c(c3)C(C)(C)c3cc(-c5ccccc5)c5ccccc5c3-4)c3cccc4c3oc3ccccc34)ccc2-c2c1cc(-c1ccccc1)c1ccccc21] | 17% |
| I-5 | If-1 | 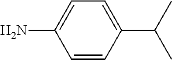 | 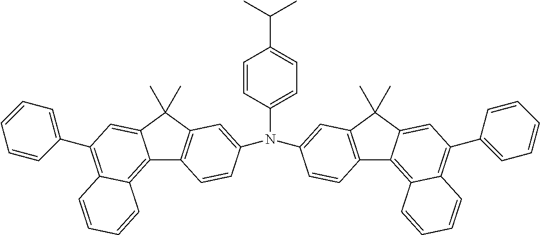 [SMILES: CC(C)c1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)cc1] | 28% |
| I-6 | If-1 | 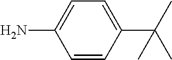 | 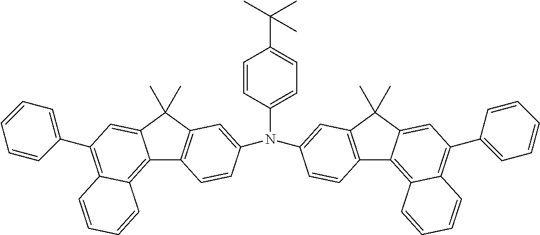 [SMILES: CC(C)(C)c1ccc(N(c2ccc3c(c2)C(C)(C)c2cc(-c4ccccc4)c4ccccc4c2-3)c2ccc3c(c2)C(C)(C)c2cc(-c4ccccc4)c4ccccc4c2-3)cc1] | 33% |
| I-7 | If-1 | 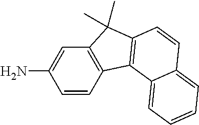 [SMILES: CC1(C)c2cc(N)ccc2-c2c1ccc1ccccc21] | 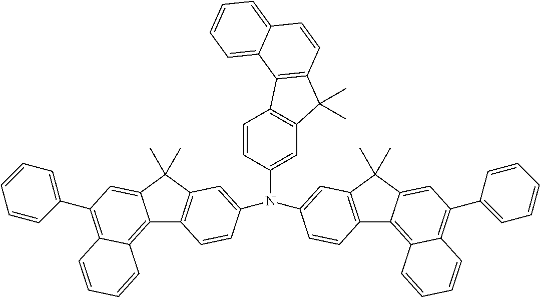 [SMILES: CC1(C)c2cc(N(c3ccc4c(c3)C(C)(C)c3cc(-c5ccccc5)c5ccccc5c3-4)c3ccc4c(c3)C(C)(C)c3cc(-c5ccccc5)c5ccccc5c3-4)ccc2-c2c1ccc1ccccc21] | 25% |
| I-8 | If-2 | 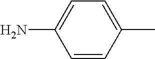 | 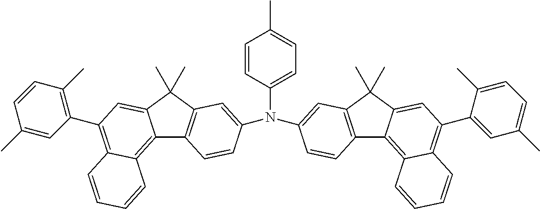 | 26% |
| I-9 | If-2 | 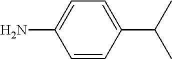 | 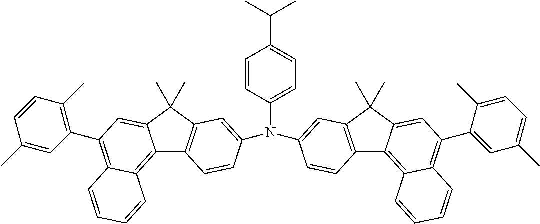 | 44% |
| I-10 | If-2 | 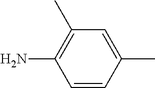 | 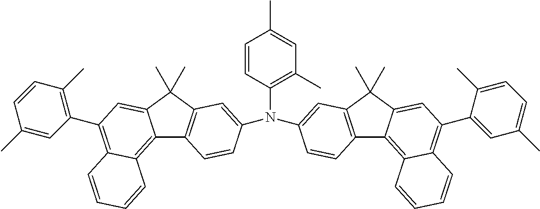 | 40% |
| I-11 | If-2 | 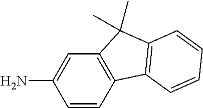 [SMILES: CC1(C)c2ccccc2-c2ccc(N)cc21] | 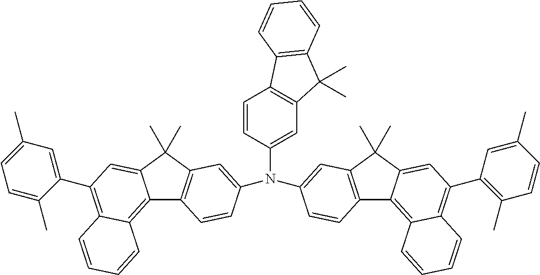 [SMILES: Cc1ccc(C)c(-c2cc3c(c4ccccc24)-c2ccc(N(c4ccc5c(c4)C(C)(C)c4ccccc4-5)c4ccc5c(c4)C(C)(C)c4cc(-c6cc(C)ccc6C)c6ccccc6c4-5)cc2C3(C)C)c1] | 21% |
| I-12 | If-3 | 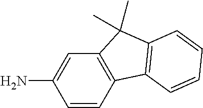 [SMILES: CC1(C)c2ccccc2-c2ccc(N)cc21] | 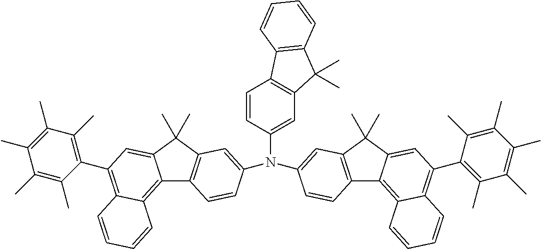 | 29% |
| I-13 | If-4 | 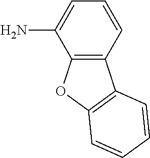 [SMILES: Nc1cccc2c1oc1ccccc12] | 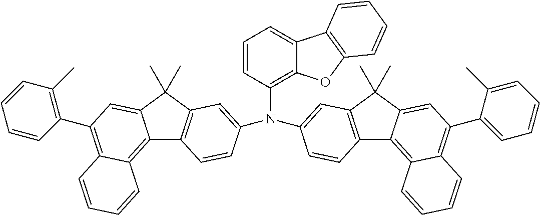 [SMILES: Cc1ccccc1-c1cc2c(c3ccccc13)-c1ccc(N(c3ccc4c(c3)C(C)(C)c3cc(-c5ccccc5C)c5ccccc5c3-4)c3cccc4c3oc3ccccc34)cc1C2(C)C] | 15% |
| I-14 | If-5 | 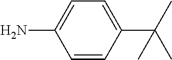 | 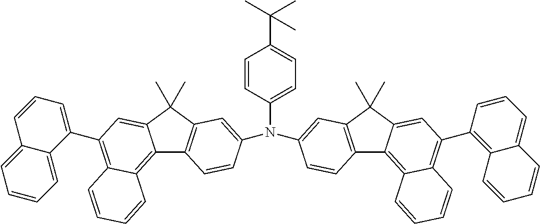 [SMILES: CC(C)(C)c1ccc(N(c2ccc3c(c2)C(C)(C)c2cc(-c4cccc5ccccc45)c4ccccc4c2-3)c2ccc3c(c2)C(C)(C)c2cc(-c4cccc5ccccc45)c4ccccc4c2-3)cc1] | 26% |
| I-15 | If-6 | 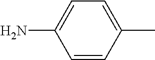 | 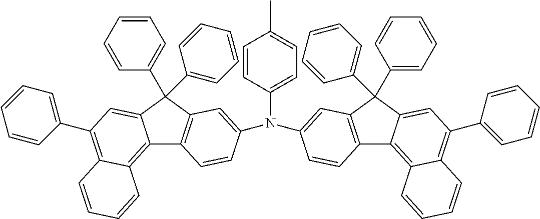 [SMILES: Cc1ccc(N(c2ccc3c(c2)C(c2ccccc2)(c2ccccc2)c2cc(-c4ccccc4)c4ccccc4c2-3)c2ccc3c(c2)C(c2ccccc2)(c2ccccc2)c2cc(-c4ccccc4)c4ccccc4c2-3)cc1] | 31% |
| I-16 | Id-7 | 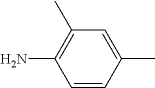 | 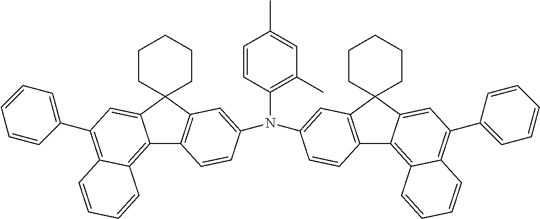 [SMILES: Cc1ccc(N(c2ccc3c(c2)C2(CCCCC2)c2cc(-c4ccccc4)c4ccccc4c2-3)c2ccc3c(c2)C2(CCCCC2)c2cc(-c4ccccc4)c4ccccc4c2-3)c(C)c1] | 29% |
| I-17 | If-8 | 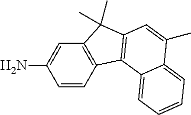 [SMILES: Cc1cc2c(c3ccccc13)-c1ccc(N)cc1C2(C)C] | 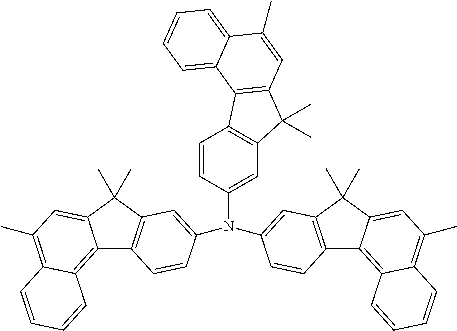 | 30% |
| I-18 | If-9 | 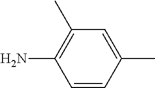 | 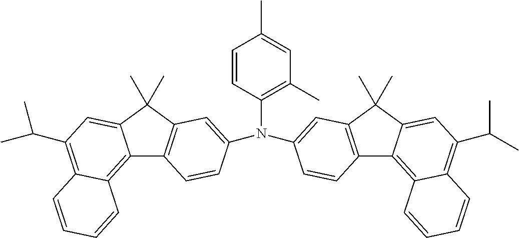 [SMILES: Cc1ccc(N(c2ccc3c(c2)C(C)(C)c2cc(C(C)C)c4ccccc4c2-3)c2ccc3c(c2)C(C)(C)c2cc(C(C)C)c4ccccc4c2-3)c(C)c1] | 28% |
| I-19 | If-10 | 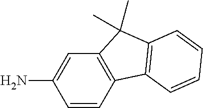 [SMILES: CC1(C)c2ccccc2-c2ccc(N)cc21] | 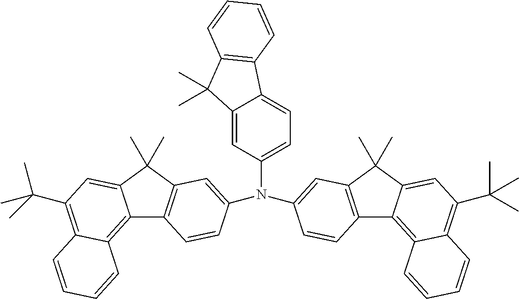 [SMILES: CC(C)(C)c1cc2c(c3ccccc13)-c1ccc(N(c3ccc4c(c3)C(C)(C)c3ccccc3-4)c3ccc4c(c3)C(C)(C)c3cc(C(C)(C)C)c5ccccc5c3-4)cc1C2(C)C] | 22% |
| I-20 | If-11 | 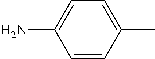 | 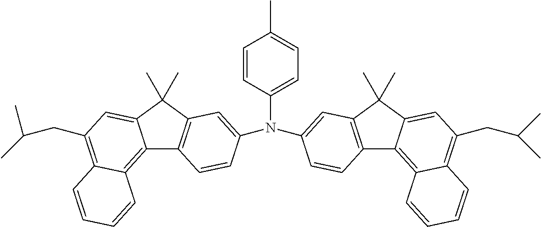 [SMILES: Cc1ccc(N(c2ccc3c(c2)C(C)(C)c2cc(CC(C)C)c4ccccc4c2-3)c2ccc3c(c2)C(C)(C)c2cc(CC(C)C)c4ccccc4c2-3)cc1] | 32% |
| I-21 | If-12 | 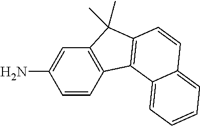 [SMILES: CC1(C)c2cc(N)ccc2-c2c1ccc1ccccc21] | 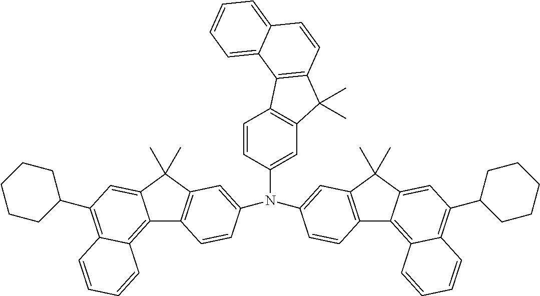 [SMILES: CC1(C)c2cc(N(c3ccc4c(c3)C(C)(C)c3cc(C5CCCCC5)c5ccccc5c3-4)c3ccc4c(c3)C(C)(C)c3cc(C5CCCCC5)c5ccccc5c3-4)ccc2-c2c1ccc1ccccc21] | 39% |

### A-2) Variant II

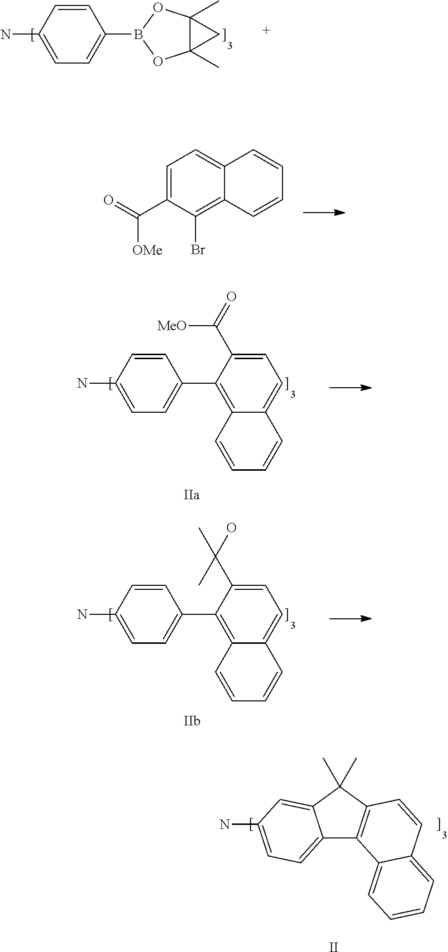 [SMILES: CC12CC1(C)OB(c1ccc(N)cc1)O2 ; COC(=O)c1ccc2ccccc2c1Br ; COC(=O)c1ccc2ccccc2c1-c1ccc(N)cc1 ; CC(C)(O)c1ccc2ccccc2c1-c1ccc(N)cc1 ; CC1(C)c2cc(N)ccc2-c2c1ccc1ccccc21 ; "II"]

The synthesis of the starting material tris-[4-(4,4,5,5-tetramethyl-1,3,2-dioxaborolan-2-yl)phenyl]amine is described in the following publication: Chem. Mater. 2011, 23, 4428-4434.

### Tris[4-(2-methoxycarbonylnaphth-1-yl)phenyl]amine (IIa)

Methyl 1-bromonaphthalene-2-carboxylate (40 g, 150 mmol), tris-[4-(4,4,5,5-tetramethyl-1,3,2-dioxaborolan-2-yl)phenyl]amine (23.5 g, 37.5 mmol) and potassium phosphate monohydrate (130 g, 0.56 mol) are dissolved in a mixture of 0.75 I of toluene, 0.75 l of dioxane and 0.75 l of water, and palladium acetate (0.67 g, 3 mmol) and tri-ortho-tolylphosphine (5.5 g, 18 mmol) are added. The batch is heated under reflux overnight, cooled to room temperature and extended with dist. water. After phase separation, the aqueous phase is extracted a number of times with toluene. The combined organic phases are washed with dist. water, dried over magnesium sulfate and filtered through AlOx. The organic phase is evaporated, giving (IIa) as a pale-yellow solid: 19.5 g (65% of theory).

### Tris-[4-(2-(propan-2-ol-2-yl)naphth-1-yl)phenyl]amine (IIb)

(IIa) (19.5 g, 25.4 mmol) and cerium(III) chloride (39 g, 81.9 mmol) are dissolved in 1 l of THF, and methylmagnesium chloride (3 M solution in THF) (330 ml, 975 mmol) is added at 0° C. The reaction solution is allowed to warm to room temperature overnight in an ice bath. The batch is carefully hydrolysed using saturated NH₄Cl solution and neutralised using 4% hydrochloric acid. The mixture is extended with dist. water and extracted thoroughly with toluene. The combined organic phases are washed a number of times with dist. water and once with sodium hydrogencarbonate solution and dried over magnesium sulfate. After removal of the solvent, the solid is recrystallised from a heptane/toluene mixture, giving 15.6 g (80% of theory) as a colourless solid.

### Tris-(7,7-dimethyl-7H-benzo[c]fluoren-9-yl)amine (II)

(IIb) (15.6 g, 19.5 mmol) is dissolved in 600 ml of DCM, and methanesulfonic acid (20 ml, 0.3 mol) and polyphosphoric acid (23 g, 230 mmol) are added at 0° C. The reaction solution is allowed to warm to room temperature overnight. The mixture is extended with ethanol and evaporated. The residue is dissolved in toluene, washed with NaOH solution and dist. water and dried over magnesium sulfate. After removal of the solvent in vacuo, the solid is purified via a silica-gel column (heptane/ethyl acetate), giving compound (II) as a yellow solid: 5.46 g (40% of theory).

### A-3) Variant III

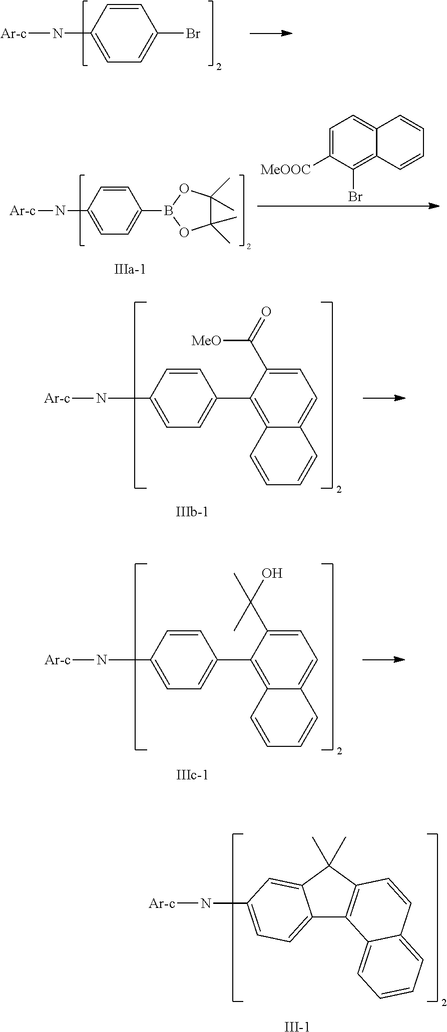

The starting materials bis-(4-bromophenyl)-(4-tert-butylphenyl)amine and bis-(4-bromophenyl)-(4-sec-butylphenyl)amine are synthesised by the method from the following publication: Journal of Polymer Science, Part A: Polymer Chemistry, 2011, vol. 49, 2, 352-360. The synthesis of biphenyl-4-yl-bis-(4-bromophenyl)amine is carried out in accordance with WO 2009/139358.

### Bis-[4-(4,4,5,5-tetramethyl-1,3,2-dioxaborolan-2-yl)phenyl]-(4-tert-butylphenyl)amine (IIIa-1)

Bis-(4-bromophenyl)-(4-tert-butylphenyl)amine (27.6 g, 60 mmol) and bispinacolatodiborane (38.1 g, 150 mmol) are dissolved in 0.5 l of THF. Potassium acetate (49 g, 500 mmol) and 1,1′-bis(diphenylphosphino)ferrocenepalladium(II) dichloride*DCM (1.45 g, 1.8 mmol) are then added to the reaction solution, which is then heated under reflux overnight. After cooling to room temperature, the batch is extended with DCM and dist. water, and the aqueous phase is extracted a number of times with DCM. The combined organic phases are washed with dist. water and, after drying using magnesium sulfate, filtered through AlOx. The solvent is removed at atmospheric pressure. The solid obtained is recrystallised from a toluene/heptane mixture, giving 23.7 g of a pale-grey powder (72% of theory).

The following compounds are prepared analogously:

| Example | | | |
|---|---|---|---|
| compound | Ar-c | Product | Yield |
| IIIa-1 | 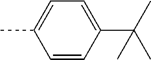 | 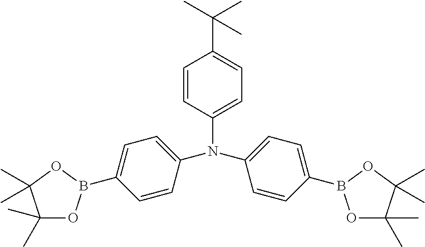 | 72% |
| IIIa-2 | 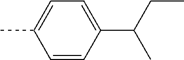 | 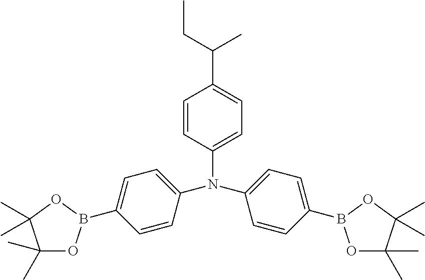 | 81% |
| IIIa-3 | 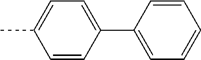 | 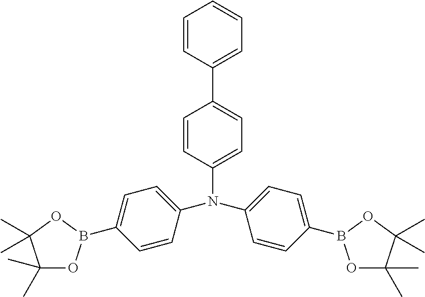 | 71% |

### Bis-[4-(2-methoxycarbonylnaphth-1-yl)phenyl]-(4-tert-butylphenyl)-amine (IIIb-1)

(IIIa-1) (23.7 g, 42.1 mmol), methyl 1-bromonaphthalene-2-carboxylate (40.0 g, 151 mmol) and potassium phosphate monohydrate (130 g, 565 mmol) are dissolved in a mixture of 0.75 l of toluene, 0.75 l of dioxane and 0.75 l of water, and palladium acetate (675 mg, 3.0 mmol) and tri-ortho-tolylphosphine (5.5 g, 18 mmol) are added. The batch is heated under reflux overnight, cooled to room temperature and extended with dist. water. After phase separation, the aqueous phase is extracted a number of times with toluene. The combined organic phases are washed with dist. water, dried over magnesium sulfate and filtered through AlOx. The organic phase is evaporated, giving compound (IIIb-1) as a pale-yellow solid: 20.6 g (73% of theory).

The following compounds are prepared analogously:

| Example | | | |
|---|---|---|---|
| compound | Ar-c | Product | Yield |
| IIIb-1 | 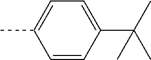 | 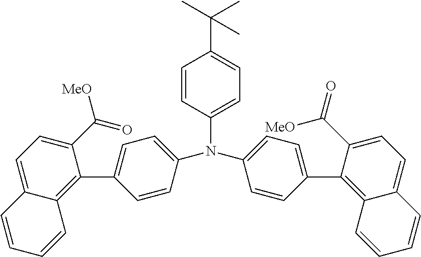 [SMILES: COC(=O)c1ccc2ccccc2c1-c1ccc(N(c2ccc(-c3c(C(=O)OC)ccc4ccccc34)cc2)c2ccc(C(C)(C)C)cc2)cc1] | 55% |
| IIIb-2 | 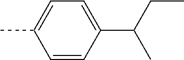 | 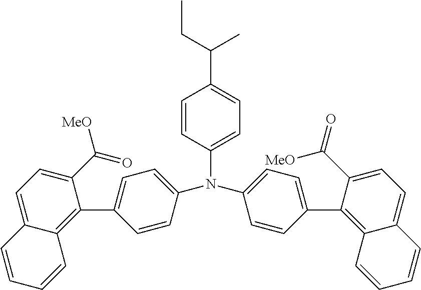 [SMILES: CCC(C)c1ccc(N(c2ccc(-c3c(C(=O)OC)ccc4ccccc34)cc2)c2ccc(-c3c(C(=O)OC)ccc4ccccc34)cc2)cc1] | 58% |
| IIIb-3 | 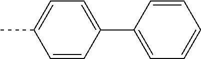 | 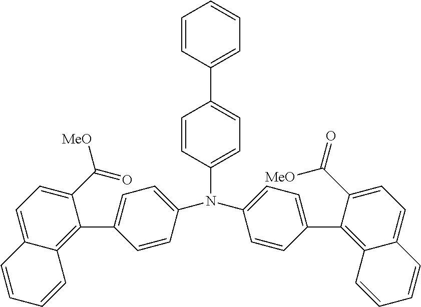 [SMILES: COC(=O)c1ccc2ccccc2c1-c1ccc(N(c2ccc(-c3ccccc3)cc2)c2ccc(-c3c(C(=O)OC)ccc4ccccc34)cc2)cc1] | 65% |

### Bis-[4-(2-(propan-2-ol-2-yl)naphth-1-yl)phenyl]-(4-tort-butylphenyl)-amine (IIIc-1)

(IIIb-1) (20.5 g, 26.7 mmol) and cerium(III) chloride (41 g, 86 mmol) are dissolved in 1 I of THF, and methylmagnesium chloride (3 M solution in THF) (350 ml, 1.05 mol) is added at 0° C. The reaction solution is allowed to warm to room temperature overnight in an ice bath. The batch is carefully hydrolysed using saturated NH₄Cl solution and neutralised using 4% hydrochloric acid. The mixture is extended with dist. water and extracted thoroughly with toluene. The combined organic phases are washed a number of times with dist. water and once with sodium hydrogencarbonate solution and dried over magnesium sulfate. After removal of the solvent, the solid is recrystallised from a heptane/toluene mixture, giving 16.4 g (80% of theory) as a colourless solid.

The following compounds are prepared analogously:

| Example | | | |
|---|---|---|---|
| compound | Ar-c | Product | Yield |
| IIIc-1 | 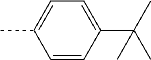 | 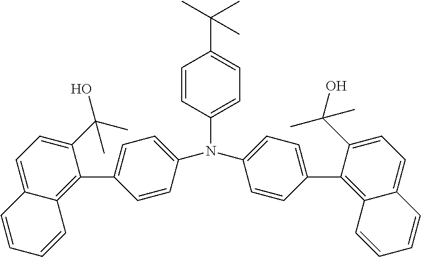 [SMILES: CC(C)(C)c1ccc(N(c2ccc(-c3c(C(C)(C)O)ccc4ccccc34)cc2)c2ccc(-c3c(C(C)(C)O)ccc4ccccc34)cc2)cc1] | 36% |
| IIIc-2 | 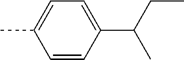 | 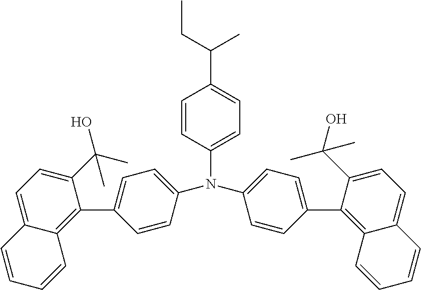 [SMILES: CCC(C)c1ccc(N(c2ccc(-c3c(C(C)(C)O)ccc4ccccc34)cc2)c2ccc(-c3c(C(C)(C)O)ccc4ccccc34)cc2)cc1] | 43% |
| IIIc-3 | 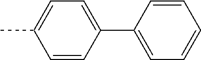 | 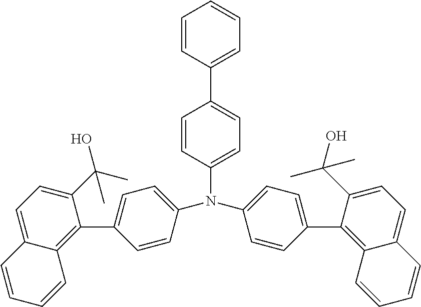 [SMILES: CC(C)(O)c1ccc2ccccc2c1-c1ccc(N(c2ccc(-c3ccccc3)cc2)c2ccc(-c3c(C(C)(C)O)ccc4ccccc34)cc2)cc1] | 41% |

### Bis-(7,7-dimethyl-7H-benzo[c]fluoren-9-yl)-(4-tert-butylphenyl)amine (III-1)

(IIIc-1) (16.2 g, 20.3 mmol) is dissolved in 600 ml of DCM, and methanesulfonic acid (20.0 ml, 300 mmol) and polyphosphoric acid (24.4 g, 24.4 mmol) are added at 0° C. The reaction solution is allowed to warm to room temperature overnight. The mixture is extended with ethanol and evaporated. The residue is dissolved in toluene, washed with NaOH solution and dist. water and dried over magnesium sulfate. After removal of the solvent in vacuo, the solid is purified via a silica-gel column (heptane/ethyl acetate) and then recrystallised from a toluene/heptane mixture, giving (III-1) as a yellow solid: 5.8 g (45% of theory).

The following compounds are prepared analogously:

| Example | | | |
|---|---|---|---|
| compound | Ar-c | Product | Yield |
| III-1 | 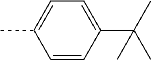 | 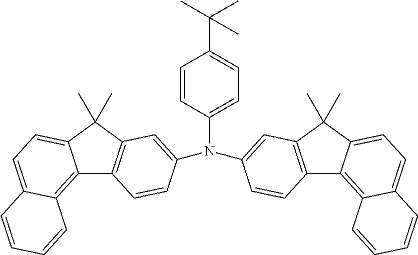 [SMILES: CC(C)(C)c1ccc(N(c2ccc3c(c2)C(C)(C)c2ccc4ccccc4c2-3)c2ccc3c(c2)C(C)(C)c2ccc4ccccc4c2-3)cc1] | 45% |
| III-2 | 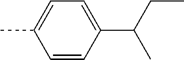 | 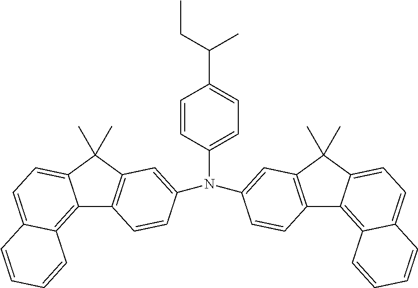 [SMILES: CCC(C)c1ccc(N(c2ccc3c(c2)C(C)(C)c2ccc4ccccc4c2-3)c2ccc3c(c2)C(C)(C)c2ccc4ccccc4c2-3)cc1] | 47% |
| III-3 | 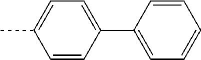 | 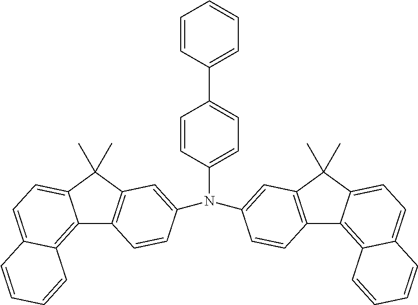 [SMILES: CC1(C)c2cc(N(c3ccc(-c4ccccc4)cc3)c3ccc4c(c3)C(C)(C)c3ccc5ccccc5c3-4)ccc2-c2c1ccc1ccccc21] | 42% |

### B) Device Examples

### Production of OLED

OLEDs according to the invention and OLEDs in accordance with the prior art are produced by a general process in accordance with WO 04/058911, which is adapted to the circumstances described here (layer-thickness variation, materials).

The data for various OLEDs are presented in the following examples (see Tables 1 to 3). The substrates used are glass plates coated with structured ITO (indium tin oxide) in a thickness of 50 nm. The OLEDs have in principle the following layer structure: substrate/buffer (20 nm)/hole-injection layer (HIL, 160 nm)/hole-transport layer (HTL, 20 nm)/emission layer (EML, 20 nm)/electron-transport layer (ETL, 30 nm)/electron-injection layer (LiQ 1 nm) and finally a cathode. The cathode is formed by an aluminium layer with a thickness of 100 nm. A layer of Clevios P VP AI 4083 (purchased from Heraeus Clevios GmbH, Leverkusen) with a thickness of 20 nm is applied as buffer by spin coating. All remaining materials are applied by thermal vapour deposition in a vacuum chamber. The structure of the EML of the OLEDs and the materials used in this layer are shown in Table 1. The structures of all materials used in the OLED are shown in Table 3, where HIL represents the material of the hole-injection layer, HTL represents the material of the hole-transport layer, ETL represents the material of the electron-transport layer and LiQ represents the material of the electron-injection layer.

The emission layer (EML) always consists of at least one matrix material (host=H) and an emitting compound (dopant=D), which is admixed with the matrix material in a certain proportion by volume by co-evaporation. An expression such as H1:D1 (95%:5%) here means that matrix material H1 is present in the layer in a proportion by volume of 95% and the emitting compound D1 is present in the layer in a proportion of 5%.

The OLEDs are characterised by standard methods. For this purpose, the electroluminescence spectra are recorded, the current efficiency (measured in cd/A) and the external quantum efficiency (EQE, measured in percent) as a function of the luminous density are calculated from current/voltage/luminous density characteristic lines (IUL characteristic lines) assuming Lambert emission characteristics, and finally the lifetime of the components is determined. The electroluminescence spectra are recorded at a luminous density of 1000 cd/m², and the CIE 1931 x and y colour coordinates are calculated therefrom. The expression EQE@1000 cd/m² denotes the external quantum efficiency at an operating luminous density of 1000 cd/m². The lifetime LT50@60 mA/cm² is the time which passes until the initial luminance (cd/m²) at a current density of 60 mA/cm² has dropped to half. The data obtained for the various OLEDs are summarised in Table 2.

### Use of the Compounds According to the Invention as Fluorescent Emitters in OLEDs

The compounds according to the invention are suitable, in particular, as blue-emitting fluorescent emitters. To this end, OLEDs comprising compounds D2, D3, D4 and D5 were produced (Examples E3 to E7).

Comparative Examples V1 and V2, in which the emitter V-D1 in accordance with the prior art is used instead of the compounds according to the invention, make it clear that a single benzofluorene substituent on an amine does not give satisfactory results (worse values for external quantum efficiency and lifetime).

By contrast, excellent values for external quantum efficiency, with deep-blue emission and an improved lifetime (LT50) are achieved with the compounds according to the invention as fluorescent emitters, as shown by Examples E3 to E7.

TABLE 1
Structure of the OLEDs

| | EML |
|---|---|
| Ex. | Thickness/nm |
| V1 | H1(95%):V-D1(5%) 20 nm |
| V2 | H2(95%):V-D1(5%)20 nm |
| E3 | H2(95%):D2(5%) 20 nm |
| E4 | H1(95%):D3(5%)20 nm |
| E5 | H2 (95%):D3(5%)20 nm |
| E6 | H2(95%):D4(5%) 20 nm |
| E7 | H2(95%):D5(5%) 20 nm |

TABLE 2
Data for the OLEDs

| | EQE | LT50 | | |
|---|---|---|---|---|
| | @ | @ | | |
| | 1000 cd/m2 | 60 mA/cm² | CIE | |
| Ex. | % | [h] | x | y |
| V1 | 2.1 | 80 | 0.15 | 0.09 |
| V2 | 2.3 | 80 | 0.16 | 0.09 |
| E3 | 5.8 | 230 | 0.15 | 0.13 |
| E4 | 6.0 | 290 | 0.15 | 0.11 |
| E5 | 6.3 | 310 | 0.15 | 0.12 |
| E6 | 7 | 320 | 0.15 | 0.11 |
| E7 | 7.4 | 340 | 0.15 | 0.12 |

TABLE 3
Structures of the materials used

| | |
|---|---|
| 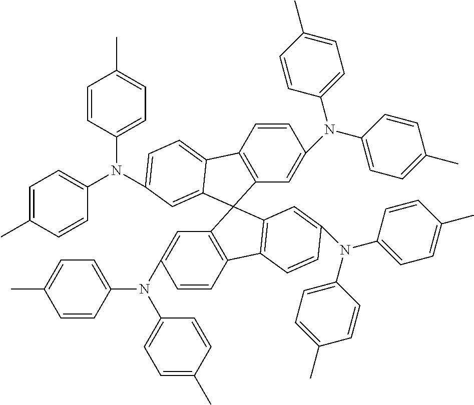 | 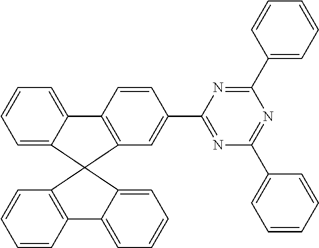 ETL |
| HIL | |
| 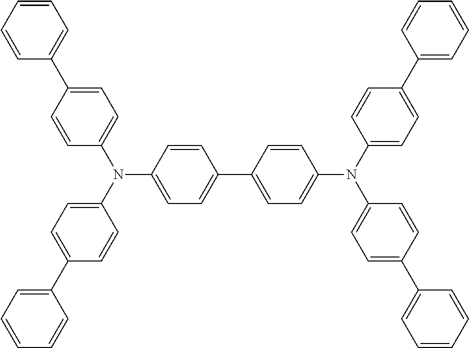 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1] | 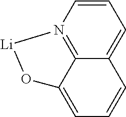 LiQ |
| HTL | |
| 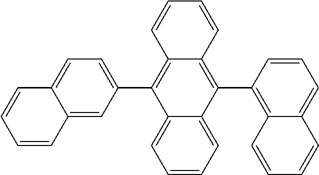 [SMILES: c1ccc2cc(-c3c4ccccc4c(-c4cccc5ccccc45)c4ccccc34)ccc2c1] | 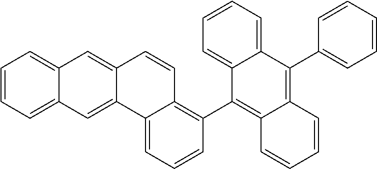 |
| H1 | H2 |
| 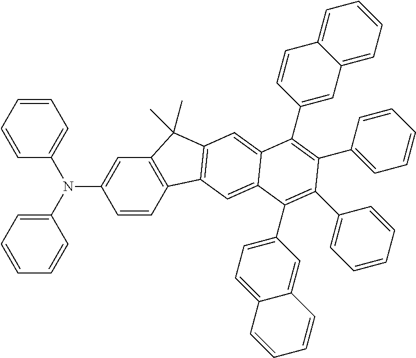 [SMILES: CC1(C)c2cc(N(c3ccccc3)c3ccccc3)ccc2-c2cc3c(-c4ccc5ccccc5c4)c(-c4ccccc4)c(-c4ccccc4)c(-c4ccc5ccccc5c4)c3cc21] | 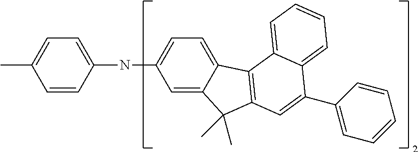 D2 (Compound I-1 of the synthesis ex.) |
| V-D1 | |
| 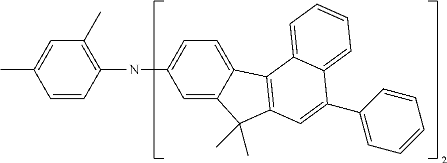 | 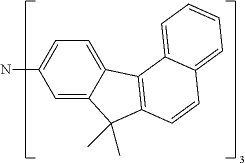 |
| D3 | D4 |
| (Compound I-3 of the synthesis ex.) | (Compound II of the synthesis ex.) |
| 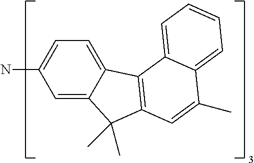 | |
| D5 | |
| (Compound I-17 of the synthesis examples) | |

1-15. (canceled)

16. A compound of formula (1) 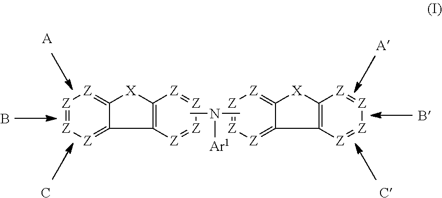
wherein
an aromatic or heteroaromatic six-membered ring, optionally substituted by one or more radicals R¹, is condensed onto at least one of the three bonds selected from the group consisting of bonds A, B, and C and onto at least one of the three bonds selected from the group consisting of bonds A′, B′, and C′;
Z is on each occurrence, identically or differently, CR¹ or N;
X is on each occurrence, identically or differently, BR², C(R²)₂, C(R²)₂—C(R²)₂, —R²C═CR²—, —R²C═N—, Si(R²)₂, Si(R²)₂—Si(R²)₂, C═O, O, S, S═O, SO₂, NR², PR², or P(═O)R²;
Ar¹ is an aromatic or heteroaromatic ring system having 6 to 40 aromatic ring atoms, optionally substituted by one or more radicals R¹;
R¹ is on each occurrence, identically or differently, H, D, F, Cl, Br, I, C(═O)R³, CN, Si(R³)₃, P(═O)(R³)₂, S(═O)R³, S(═O)₂R³, a straight-chain alkyl or alkoxy group having 1 to 20 C atoms or a branched or cyclic alkyl or alkoxy group having 3 to 20 C atoms or an alkenyl or alkynyl group having 2 to 20 C atoms, wherein the said groups are each optionally substituted by one or more radicals R³, and wherein one or more CH₂ groups in said groups are optionally replaced by —R³C═CR³—, —C≡C—, Si(R³)₂, C═O, C═NR³, —C(═O)O—, —C(═O)NR³—, NR³, P(═O)(R³), —O—, —S—, SO, or SO₂ and wherein one or more H atoms in said groups are optionally replaced by D, F, Cl, Br, I, or CN, an aromatic or heteroaromatic ring system having 5 to 30 aromatic ring atoms, optionally substituted by one or more radicals R³, or an aryloxy or heteroaryloxy group having 5 to 30 aromatic ring atoms, optionally substituted by one or more radicals R³, and wherein two or more radicals R¹ are optionally linked to one another so as to define a ring;
R² is on each occurrence, identically or differently, H, D, F, Cl, Br, I, C(═O)R³, CN, Si(R³)₃, N(R³)₂, P(═O)(R³)₂, S(═O)R³, S(═O)₂R, a straight-chain alkyl or alkoxy group having 1 to 20 C atoms or a branched or cyclic alkyl or alkoxy group having 3 to 20 C atoms or an alkenyl or alkynyl group having 2 to 20 C atoms, wherein said groups are each optionally substituted by one or more radicals R³, and wherein one or more CH₂ groups in said groups are optionally replaced by —R³C═CR³—, —C≡C—, Si(R³)₂, C═O, C═NR³, —C(═O)O—, —C(═O)NR³—, NR³, P(═O)(R³), —O—, —S—, SO, or SO₂, and wherein one or more H atoms in said groups are optionally replaced by D, F, Cl, Br, I, or CN, an aromatic or heteroaromatic ring system having 5 to 30 aromatic ring atoms, optionally substituted by one or more radicals R³, or an aryloxy or heteroaryloxy group having 5 to 30 aromatic ring atoms, optionally substituted by one or more radicals R³, and wherein two or more radicals R² are optionally linked to one another so as to define a ring;
R³ is on each occurrence, identically or differently, H, D, F, Cl, Br, I, C(═O)R⁴, CN, Si(R⁴)₃, N(R⁴)₂, P(═O)(R⁴)₂, S(═O)R⁴, S(═O)₂R⁴, a straight-chain alkyl or alkoxy group having 1 to 20 C atoms or a branched or cyclic alkyl or alkoxy group having 3 to 20 C atoms or an alkenyl or alkynyl group having 2 to 20 C atoms, where said groups are each optionally substituted by one or more radicals R⁴, and wherein one or more CH₂ groups in said groups are optionally replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO, or SO₂, and wherein one or more H atoms in said groups are optionally replaced by D, F, Cl, Br, I, or CN, an aromatic or heteroaromatic ring system having 5 to 30 aromatic ring atoms, optionally substituted by one or more radicals R⁴, or an aryloxy or heteroaryloxy group having 5 to 30 aromatic ring atoms, optionally substituted by one or more radicals R⁴, and wherein two or more radicals R¹ are optionally linked to one another so as to define a ring;
R⁴ is on each occurrence, identically or differently, H, D, F, or an aliphatic, aromatic or heteroaromatic organic radical having 1 to 20 C atoms, wherein one or more H atoms are optionally replaced by D or F; and wherein two or more substituents R⁴ are optionally linked to one another so as to define a ring;
with the proviso that compounds having an aromatic or heteroaromatic six-membered ring is condensed onto each of the two bonds A and A′ but no further aromatic or heteroaromatic six-membered ring condensed onto one of the other four bonds selected from the group consisting of bonds B, C, B′, and C′ are excluded.

17. The compound of claim 16, wherein no aromatic or heteroaromatic six-membered ring is condensed onto either of bonds A and A′.

18. The compound of claim 16, wherein an aromatic or heteroaromatic six-membered ring is in each case condensed onto at least one of the two bonds selected from the group consisting of bonds B and C and onto at least one of the two bonds selected from the group consisting of bonds B′ and C′, wherein the aromatic or heteroaromatic six-membered ring is optionally substituted by one or more radicals R′.

19. The compound of claim 16, wherein X is selected on each occurrence, identically or differently, from the group consisting of BR², C(R²)₂, Si(R²)₂, O, S, and NR².

20. The compound of claim 16, wherein Z is CR¹.

21. The compound of claim 16, wherein Ar¹ is selected from the group consisting of phenyl, naphthyl, biphenyl, terphenyl, fluorenyl, monobenzofluorenyl, dibenzofluorenyl, spirobifluorenyl, indenofluorenyl, dibenzofuranyl, carbazolyl, and dibenzothiophenyl, each of which is optionally substituted by one or more radicals R¹.

22. The compound of claim 16, wherein the compound is a compound of formula (I-A):

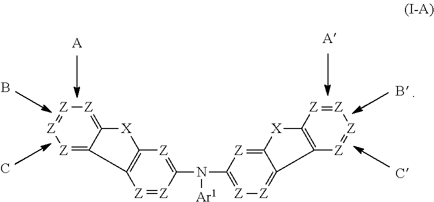

23. The compound of claim 16, wherein the compound contains no further arylamino group in addition to the arylamino group —N(Ar¹)—.

24. The compound of claim 16, wherein the compound is a compound of formulae (I-1) to (I-5): 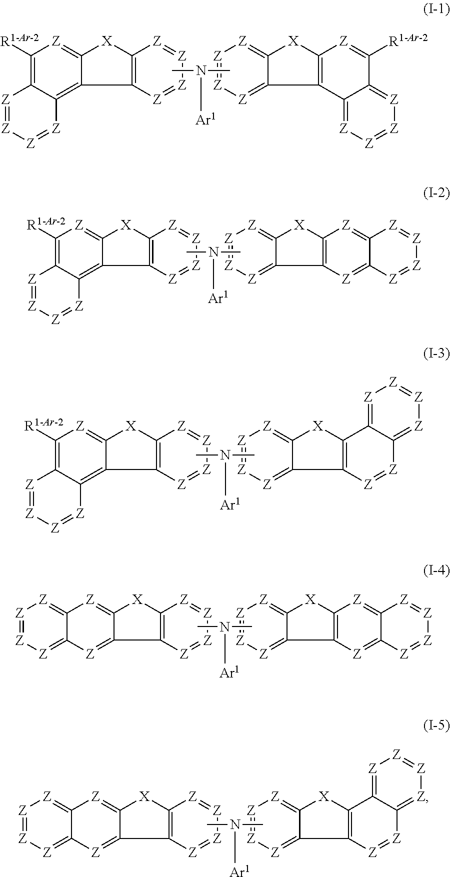
wherein
R^{1-Ar-2}
is selected on each occurrence, identically or differently, from the group consisting of H or straight-chain alkyl groups having 1 to 10 C atoms or branched or cyclic alkyl groups having 3 to 10 C atoms, optionally substituted by one or more radicals R³, or aromatic or heteroaromatic ring systems having 6 to 18 aromatic ring atoms, optionally substituted by one or more radicals R³.

25. The compound of claim 24, wherein the X is C(R²)₂.

26. An oligomer, polymer, or dendrimer comprising one or more compounds of claim 16, wherein the bond(s) to the polymer, oligomer, or dendrimer may be localised at any positions in formula (I) substituted by R¹ or R².

27. A formulation comprising at least one compound of claim 16 and at least one solvent.

28. A formulation comprising at least one polymer, oligomer, or dendrimer of claim 26 and at least one solvent.

29. An electronic device comprising at least one compound of claim 16.

30. The electronic device of claim 29, wherein the electronic device is selected from the group consisting of organic integrated circuits, organic field-effect transistors, organic thin-film transistors, organic light-emitting transistors, organic solar cells, organic optical detectors, organic photoreceptors, organic field-quench devices, organic light-emitting electrochemical cells, organic laser diodes, and organic electroluminescent devices.

31. The electronic device of claim 30, wherein the electronic device is an organic electroluminescent device and wherein the at least one compound is present in at least one organic layer selected from the group consisting of hole-transport layer, hole-injection layer, electron-blocking layer, and emitting layer.
